(19) United States
(12) Patent Application Publication
(10) Pub. No.: US 2022/0204626 A1
(43) Pub. Date: Jun. 30, 2022

(54) BISPECIFIC FUSION PROTEIN AND APPLICATION THEREOF

(71) Applicant: Remegen Co., Ltd., Yantai, Shandong (CN)

(72) Inventor: Jianmin Fang, Yantai District, Shandong (CN)

(21) Appl. No.: 17/594,768

(22) PCT Filed: Mar. 19, 2021
(86) PCT No.: PCT/CN2021/081659

§ 371 Date: Oct. 28, 2021

(30) Foreign Application Priority Data
Mar. 20, 2020 (CN) 202010199036.2

(51) Int. Cl. C07K 16/28; C12N 15/62; C12N 15/63; G01N 33/574; C07K 19/00; A61P 35/00

(52) U.S. Cl. CPC C07K 16/2827; C12N 15/62; A61P 35/00; G01N 33/574; C07K 19/00; C12N 15/63

(57) ABSTRACT

Provided is a bispecific fusion protein having a novel structure. A second binding structural domain is inserted into an IgG hinged region in a full length by means of an optional peptide joint. The fusion protein has the same expression and production advantages as those of IgG, does not influence the binding activity of an Fab region of the fusion protein and further improves the stability and obtains a higher half life. In addition, the binding activity of the second binding structural domain to a target binding site is significantly improved with respect to the binding activity of a monomer corresponding to a soluble natural binding fragment to a corresponding target.

### FIELD

The present disclosure relates to a bispecific fusion protein with a novel structure and application thereof. The present disclosure also relates to a polynucleotide, a nucleic acid construct and a host cell encoding or preparing the fusion protein.

### BACKGROUND

With the development of biotechnology, antibodies, as mature therapeutic drugs, play an important role in the treatment of tumors and immune diseases. Currently, most of the antibody drugs on the market target a single antigen and can only act on one target molecule. However, complex diseases are often multifactorial in nature, involving the overexpression or synergy of disease mediators, or the upregulation of different receptors, including molecular interactions in their signaling networks. Therefore, regulating multiple signal pathways can improve the efficacy of therapeutic antibodies. Bispecific antibody (BsAb) therapy using dual targeting strategies has become one of the effective treatments for tumors and immune diseases (Kontermann R. Dual targeting strategies with bispecific antibodies [C]//MAbs. Taylor & Francis, 2012, 4(2): 182-197.).

Bispecific antibodies can specifically bind to two different antigens or epitopes (Carter P. Bispecific human IgG by design [J]. Journal of immunological methods, 2001, 248(1-2): 7-15.). As early as the 1980s, Morrison et al. first prepared the first truly tetravalent bispecific antibody against dextran and dansyl group by linking single-chain antibodies with different specificities using flexible peptides for fusion expression (Colonia M J, Morrison S L. Design and production of novel tetravalent bispecific antibodies W. Nature biotechnology, 1997, 15(2): 159.). However, due to the bottleneck of antibody preparation technology and the lack of basic research on signal pathways, the development of bispecific antibodies has been hindered. With the in-depth understanding of disease pathogenesis and the rapid development of therapeutic monoclonal antibodies (McAbs)-related technologies, the technologies of antibody's construction, expression and purification has greatly improved, enabling the development of bispecific antibodies has the technology and motivation to overcome the limiting factors. At present, the mechanisms of bispecific or multispecific antibodies are mainly as follows: (1) bridging cells (in trans); (2) receptor inhibition (in-cis) or receptor activation (in-cis); (3) co-factor mimetic; (4) PIgGyback approaches (Aran F. Labriin, et al. Bispecific antibodies: a mechanistic review of the pipeline, Nature Reviews Drug Discovery volume 18, pages 585-608(2019)).

In the development of bispecific antibodies, the choice of molecular structure is very important. Different bispecific antibody designs have their own advantages and disadvantages, but the design of bispecific antibodies for clinical treatment purposes must solve the following problems: first, ensuring the correct coupling or pairing of two pairs (or more) of different light chains and heavy chains; second, maintaining the independence of each binding domain of each monoclonal antibody, and when binding different epitopes at the same time, there will be no steric interference between each other; third, the antibody molecules should be easy to express in mammalian cells without complicated protein modification process; fourthly, good druggability is required, such as high thermal stability, high chemical stability, high solubility, not easy to polymerize, low viscosity, high expression, suitable half-life, and the like (Spiess C, Zhai Q, Carter P J. Alternative molecular formats and therapeutic applications for bispecific antibodies [J]. Molecular immunology, 2015, 67(2): 95-106.).

According to different molecular structure forms (i.e., components and construction methods), bispecific antibodies can be divided into many types. For example, according to the left-right symmetry of the structure, they are divided into symmetrical and asymmetrical structures; according to the presence or absence of Fc region, they can be divided into bispecific antibodies containing Fc region and bispecific antibodies devoid of Fc region; and according to the number of antigen-binding regions, they are divided into bivalent, trivalent, tetravalent or multivalent configurations, etc. At present, bispecific antibodies have developed dozens of structures (Spiess C, Zhai Q, Carter P J. Alternative molecular formats and therapeutic applications for bispecific antibodies [J]. Molecular immunology, 2015, 67(2): 95-106.), these dozens of bispecific antibodies have diverse structures, roughly divided into 5 categories, including IgG-like bispecific antibodies, IgG-like bispecific antibodies with additional functional regions, bispecific antibodies with different antigen-binding fragments linked in series, fusion protein-type bispecific antibodies, and chemically coupled bispecific antibodies. Among them, the construction of IgG-like bispecific antibodies mainly employs recombinant DNA technology for structural modification. The constructed IgG-like bispecific antibodies have complete crystallizable fragments (i.e., Fc fragments), and thus they can bind to different antigens while still retaining Fc fragment-mediated ADCC (antibody-dependent cell-mediated cytotoxicity) and CDC (complement-mediated cytotoxicity, complement-dependent cytotoxicity) and the like, and at the same time, this type of antibody also retains the feature of extending the in vivo half-life of the antibody by binding to the neonatal Fc receptor (FcRn) (Ridgway J B B, Presta. L G, Carter P. ‘Knobs-into-holes’ engineering of antibody CH3 domains for heavy chain heterodimerization [J]. Protein Engineering, Design and Selection, 1996, 9(7): 617-621.); IgG-like bispecific antibodies with additional functional regions are generally based on traditional IgG antibodies and generated by adding other specific antigen-binding fragments (i.e., additional functional regions, such as single domain antibodies, single chain antibodies, etc.) to the heavy chain and/or light chain by means of fusion proteins (LaFleur D, Abramyan D, Kanakaraj P, et al. Monoclonal antibody therapeutics with up to five specificities: functional enhancement through fusion of target-specific peptides [C]//MAbs. Taylor & Francis, 2013, 5(2): 208-218.); bispecific antibodies with different antigen-binding fragments linked in series are obtained by linking different Fabs, single-chain antibodies, single-domain antibodies, or antigen-binding fragments in a certain sequence through a linking peptide (Stork R, Müller D, Kontermann R E. A novel tri-functional antibody fusion protein with improved pharmacokinetic properties generated by fusing a bispecific single-chain diabody with an albumin-binding domain from streptococcal protein G [J]. Protein Engineering, Design & Selection, 2007, 20(11): 569-576.); fusion protein-type bispecific antibodies are protein molecules that can bind two or more antigens at the same time, formed by linking antibodies/antibody fragments (IgG, Fab, scFv, etc.) with different specificities through a linker such as a polypeptide fragment; and chemically coupled bispecific antibodies are similar to fusion protein-type bispecific antibodies, while for the chemically coupled bispecific antibodies, two antibodies/antibody fragments must be prepared separately first, and then the two are coupled through chemical bonds or both are coupled to a carrier protein.

In addition, with the development of basic research on signal pathways and the expansion of hi-antibody mechanisms, there are more and more targets and combinations available for bispecific antibody selection, such as PD-1, PD-L1, CTLA-4, LAG-3, FGL1, TIM-3, Galectin-9, CD155 and TGF-β receptor (TGF-βR), CD80, CD86, VEGFR, VEGF-trap, FGL1, CD70, 4-1BBL, OX40L, and SIRPα; and tumor antigens including Claudin 18.2, HER-2, Mesothelin, BCMA, SSTR2, GPRC5D, PSMA, FCRH5, CD33, CD123, CD20, A33, CEA, CD28, DLL3, EGFR, VEGFR, VEGFR2, VEGF-A, Nectin-4, FGAR, C-met, RANKL, PDGFR, PDGFRα, DLL4, Ang-1, and Ang-2, etc. Combinations of immune checkpoint targets or tumor targets with other targets relating to resisting the development of cancer cells or activating T-cells, or the combination of multiple immune checkpoint targets greatly improves the possibility of treating malignant diseases such as tumors. In the early stage, the combination of two separate checkpoint antibodies was used clinically to improve the efficacy. For example, compared with treatment with ipilimumab alone, the treatment with the combination of ipilimumab (anti-CTLA4) and nivolumab (anti-PD1) can improve the survival rate of patients with melanoma. Currently, the safety and early efficacy of the four PD-1×CTLA4 bsAbs are being evaluated in early clinical trials. The concept of inhibitors blocking two immune checkpoints is also used clinically for other target combinations, such as PD-1×LAG 3, PD-1×TIM3, and PD-L1×CTLA 4. (Wolchok, J. D. et al. Overall Survival with Combined Nivolumab and Ipilimumab in Advanced Melanoma, N. Engl. J. Med. 377(14): 1345-1356. (2017); Dovedi, S. J. et al. MED15752: A novel bispecific antibody that preferentially targets CTLA-4 on PD-1 expressing T-cells. Cancer Res. 78(13), Supplement. Abstract 2776: (2018). Hedvat, M. et al. Simultaneous checkpoint—checkpoint or checkpoint—costimulatory receptor targeting with bispecific antibodies promotes enhanced human T cell activation [abstract P664]. Presented at the 2018 Society for Immunotherapy of Cancer (SITC) (2018); Aran F. Labrijn, et al. Bispecific antibodies: a mechanistic review of the pipeline, Nature Reviews Drug Discovery volume 18, pages 585-608(2019).

At present, most of the bispecific antibodies are still in the clinical or preclinical research stage, and only three bispecific antibodies have been approved for marketing. They are Trion Pharma's Catumaxomab (delisted due to commercial factors in 2017), Amgen's Blinatumomab and Roche's Emicizumab, Catumaxomab is an IgG-like bispecific antibody in the form of Triomab. It targets T cells and tumor cells on which epithelial cell adhesion molecules (EpCAMs) overexpress, and inhibits tumor cells through T cell activation, antibody-dependent cell-mediated cytotoxicity (ADCC), and complement-dependent cytotoxicity (CDC). It was approved by EMA in 2009 for the treatment of malignant ascites caused by EpCAM-positive tumors that were ineffective or unfeasible against the standard treatment, and it is also the first bispecific antibody to be approved for marketing. Blinatumomab is a bispecific antibody without Fc region and adopts the structure form of BITE (bispecific T cell engager). Blinatumomab binds to T cells through the anti-CD3 part, and to malignant and normal B cells through the anti-CD19 part, thereby inducing T cell-mediated tumor cell killing effect. In 2014, it was approved by FDA for the treatment of Philadelphia chromosome-negative precursor B-cell acute lymphoblastic leukemia. Emicizumab is a modified humanized bispecific IgG4 monoclonal antibody that can bind to factor IXa and factor X. This antibody adopts “Knobs-into-Holes (KiH)” for Fc design, “Common Light Chain-IgG (CLC-IgG)” for LC design, and “Multidimensional Optimization” for variable region optimization, and was approved by FDA in 2017 for routine prevention of hemophilia Ain the presence of factor VIII inhibitors.

TABLE 1
Bispecific antibodies that have been
approved for marketing (including delisted)

| Bispecific antibody | Trade name | Research unit | Time of first approval |
|---|---|---|---|
| Catumaxomab | Removab | Trion Pharma | 2009 (EMA) |
| | | | (delisted in 2017) |
| Blinatumomab | Blincyto | Amgen | 2014 (FDA) |
| Emicizumab | Hemlibra | Roche | 2017 (FDA) |

Although there are currently dozens of bispecific antibodies/platforms and research and development experience from 3 marketed drugs, there are more problems and challenges in the development of bispecific antibodies compared to general antibodies due to their complex structures and diverse functional characteristics. For example, IgG-like bispecific antibodies face the problem of mismatches of heavy chain-heavy chain or heavy chain-light chain during the preparation process, the most typical of which is Catumaxomab. Although Catumaxomab is the first bispecific antibody approved for marketing, there are some very obvious limitations, which are mainly reflected in the complex production process of Triomab antibody and the immunogenicity problem that heterologous antibodies are relatively prone to emerge, and in 2017, the drug was delisted due to commercial factors; for another example, bispecific antibodies with different antigen-binding fragments linked in series have a short half-life due to the lack of constant regions, especially the lack of Fc fragments, and their expression level and intracellular stability after expression are generally lower than traditional IgG (Stork R, Muller D, Kontermann R E. A novel tri-functional antibody fusion protein with improved pharmacokinetic properties generated by fusing a bispecific, single-chain diabody with an albumin-binding domain from streptococcal protein G [J]. Protein Engineering, Design & Selection, 2007, 20(11): 569-576.). In addition, most of this type of antibody is difficult to adopt affinity purification means in the purification process, so the purification process and the analysis of impurities in the finished product are also more complicated than traditional IgG (Tan P Sandmaier B M, Stayton P S. Contributions of a highly conserved VH/VL hydrogen bonding interaction to scFv folding stability and refolding efficiency [J]. Biophysical journal, 1998, 75(3): 1473-1482.), the most typical of which is Blinatumomab. Blinatumomab has a small molecular weight because of lacking Fc fragments, and its half-life in the blood is only 1.25±0.63 h, which is much shorter than the half-life of an intact antibody, so it requires continuous intravenous injection for 4 weeks to reach the effective dose, and the reagent needs to be additionally equipped with a continuous infusion device when used. Besides, it has poor stability and easily forms aggregates during production, which also affects the quality of the product and increases production costs. The current bi specific antibodies have many problems, such as low expression, poor stability, complex production processes, and significantly higher research and development costs than monoclonal antibodies, all of which limit the development of bispecific antibodies. Therefore, there is an urgent need to develop bispecific antibodies with new structures to provide more clinical options.

### SUMMARY

In response to the above problems, the present disclosure provides a bispecific fusion protein with a novel structure, which belongs to an IgG-like bispecific fusion protein with additional functional regions. Specifically, the fusion protein has a structure in which a second binding domain is inserted in the hinge region of a full-length immunoglobulin G (IgG) through an optional peptide linker, and the Fab region of the IgG is a first binding domain of the fusion protein; after inserting the second binding domain, the heavy chain of the IgG is the heavy chain of the fusion protein, and the light chain of the IgG is the light chain of the fusion protein; the second binding domain is selected from a human receptor or ligand, an extracellular region fragment of the receptor or ligand, a binding domain fragment, and a fragment combination of the receptor or ligand, wherein the receptor or ligand forms a dimerization or multimerization structure in a natural signaling pathway to activate or inhibit the signaling pathway, and the second binding domain and the first binding domain target different targets. The bispecific fusion protein with the above structure was named as Hibody (Hinge-insertion bispecific antibody) structure fusion protein (hereinafter referred to as Hibody structure fusion protein or Hibody).

Further, the first binding domain targets and binds to an immune checkpoint molecule or a tumor antigen.

Further, the immune checkpoint molecule includes PD-1 (Programmed cell death protein 1), PD-L1 (Programmed cell death 1 ligand 1), CTLA-4 (Cytotoxic T lymphocyte-associated antigen-4), LAG-3 (Lymphocyte activation gene-3), FGL1 (Fibrinogen-like protein 1), TIM-3 (T cell immunoglobulin-3), Galectin-9, TIGIT (T-cell immunoreceptor with Ig and ITIM domains), CD155, CD47; the tumor antigen includes Claudin 18.2, Her-2 (Human epidermal growth factor receptor-2), Mesothelin, BCMA (B Cell Maturation Antigen), SSTR2 (Somatostatin receptor 2), GPRC5D (G-protein coupled receptor family C group 5 member), PSMA (Prostate specific membrane antigen), FcRH5 (Fe receptor-like protein 5), CD33, CD123, CD20, A33, CEA (Carcino-embryonic antigen), CD28, DLL3 (Delta-like protein 3), EGFR (Epidermal growth factor receptor), VEGFR (Vascular Endothelial Growth Factor Receptor), VEGFR2 (Vascular Endothelial Growth Factor Receptor 2), VEGF-A (Vascular endothelial growth factor-A), Nectin-4, FGFR (Fibroblast growth factor receptors), C-met, RANKL (Receptor activator of nuclear factor kappa-B ligand), PDGF (Platelet Derived Growth Factor), PDGFR (Platelet Derived Growth Factor Receptor), PDGFRα (Platelet Derived Growth Factor Receptor α), DLL4 (Delta-like ligand 4), Ang-1 (Angiopoietin-1), and Ang-2 (Angiopietin-1).

Further, the second binding domain targets and binds to human TGF-β (Transforming growth factor-β), CTLA-4, VEGF, LAG3, CD27, 4-1BB (CD137), OX40 (CD134), CD47, FGL1 (Fibrinogen Like Protein 1), TLT-2 (Trem-like transcript 2), CD28, HGF (Hepatocyte growth factor), CSF1 (Colony-stimulating factor 1), CXCL1 (CXC chemokine ligand 1), CXCL2 (CXC chemokine ligand 2), CXCL3 (CXC chemokine ligand 3), CXCL5 (CXC chemokine ligand 5), CXCL6 (CXC chemokine ligand 6), CXCL7 (CXC chemokine ligand 7), CXCL8 (CXC chemokine ligand 8), CXCL9 (CXC chemokine ligand 9), CXCL10 (CXC chemokine ligand 10), CXCL12 (CXC chemokine ligand 12), GITR (Glucocorticoid-induced tumor necrosis factor receptor), EGF (Epidermal Growth Factor), and ICOSL (inducible co-stimulator ligand).

Further, the receptor or ligand is human TGF-β receptor (TGF-βR), CD80, CD86, VEGFR, VEGF-trap, FGL1, CD70, 4-1BBL, OX40L, SIRPα (Signal regulatory protein α), B7-H3 (CD276), C-met, CSF1R (Colony-stimulating factor 1 receptor), CXCR2 (CXC chemokine receptor 2), CXCR3 (CXC chemokine receptor 3), CXCR4 (CXC chemokine receptor 4), GITRL (Glucocorticoid-induced TNF receptor ligand), EGFR, and ICOS (inducible co-stimulator).

Among them, CD80 and CD86 are transmembrane glycoproteins, which are members of the immunoglobulin superfamily (IgSF). The mature CD80 molecule consists of 254 amino acids, including 208 amino acids in the extracellular domain (ECD), 25 amino acids in the transmembrane domain, and 21 amino acids in the intracellular domain. Similarly, the mature CD86 molecule consists of 303 amino acids, including 222 amino acids in the extracellular domain, 20 amino acids in the transmembrane domain, and 61 amino acids in the intracellular domain. The extracellular domains of CD80 and CD86 include immunoglobulin V (IgV) and immunoglobulin C (IgC) regions. CD80 and CD86 bind to their ligands CD28 and CTLA-4 through the immunoglobulin V (IgV) region. In the case of CD80 and CD86 binding to CD28, CD80 and CD86 have important regulatory effects on antigen-induced T cell activation, proliferation, and production of effector function, acting as positive regulators; while in the case of CD80 and CD86 binding to CTLA-4, CD80 and CD86 down-regulates the immune response, acting as negative regulators. Therefore, CD80 and CD86 are co-stimulatory factors when T lymphocytes are activated, and they play an important role in autoimmune monitoring, humoral immune response and transplantation response. Different from using agonistic anti-CD28 antibodies to activate T cells, using CD28's ligand CD80 as an activating factor to activate T cells does not trigger a severe cytokine storm, thereby greatly reducing the possibility of endangering the life of the patient.

In some embodiments, CD80 ECD is selected from human CD80 (such as the human CD80 of SEQ ID NO: 134) or human CD80 ECD derived from CD80 isotype 2 or isotype 3 (SEQ ID NO: 135 and 136). The CD80 ECD comprises CD80 immunoglobulin V (IgV) region (CD80-IgV, SEQ ID NO: 133). In one embodiment, the CD80 ECD comprises human CD80 immunoglobulin V region and C region (CD80-IgVIgC, SEQ ID NO: 32). In one embodiment, the CD80 ECD is human CD80 ECD. In one embodiment, the CD80 ECD comprises human CD80 IgV.

| CD80_IgV (SEQ ID NO: 133): | |
|---|---|
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADFPT PS | 112 |
| CD80-IgVIgC (SEQ ID NO: 32): | |
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADFPT PSISDFEIPT | 120 |
| SNIRRIICST SGGFPEPHLS WLENGEELNA INTTVEQDPE TELYAVSSKL DFNMTTNHSF | 180 |
| MCLIKYGHLR VNQTFNWN | 198 |
| CD80 isotype 1 (SEQ ID NO: 134): | |
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD ECTYECVVLK | 120 |
| YEKDAFKREH LAEVTLSVKA DFPTPSISDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNLLPSWAIT LISVNGIFVI CCLTYCFAPR CRERRRNERL RRESVRPV | 288 |
| CD80 isotype 2 (SEQ ID NO: 135): | |
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DFPTPSISDF EIPTSNIRRI ICSTSGGFPE PHLSWLENGE | 180 |
| ELNAIMTTVS QDPETELYAV SSKLDFNMTT NHEFMCLIKY GHLRVNQTFN WNTSFAPRCR | 240 |
| ERRRNERLRR ESVRPV | 256 |
| CD80 isotype 3 (SEQ ID NO: 136): | |
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKG FAPRCRERRR NERLRRESVR PV | 162 |

Further, the first binding domain and the second binding domain are selected from the following combinations:

(1) The first binding domain targets and binds to PD-ti, and the second binding domain targets and binds to human TGF-β, VEGF, FGL1, CD47, CD155, HGF. CSF1, CXCL1, CXCL2, CXCL3, CXCL5, CXCL6, CXCL7, CXCL8, CXCL9, CXCL10, CXCL12, EGF or ICOSL; or

(2) The first binding domain targets and binds to PD-1, and the second binding domain targets and binds to human CTLA-4, VEGF, HGF, EGF, CD28, LAG-3, CD27, 4-1BB or OX40; or

(3) The first binding domain targets and binds to CTLA-4, and the second binding domain targets and binds to human CD28, VEGF, HGF, EGF, LAG-3, CD27, 4-1BB or OX40; or

(4) The first binding domain targets and binds to LAG-3, and the second binding domain targets and binds to human CD28, VEGF, HGF, EGF, CTLA-4, CD27, 4-1BB or OX40; or

(5) The first binding domain targets and binds to and the second binding domain targets and binds to human TGF-3, VEGF, CD47, CD155, HGF, CSF1, CXCL1, CXCL2, CXCL3, CXCL5, CXCL6, CXCL7, CXCL8, CXCL9, CXCL10, CXCL12, EGF or ICOSL; or

(6) The first binding domain targets and binds to TIM-3, and the second binding domain targets and binds to human VEGF, HGF, EGF, LAG3, CD27, 4-1BB or OX40; or

(7) The first binding domain targets and binds to Galectin-9, and the second binding domain targets and binds to human TGF-β, VEGF, CD47, CD155, HGF, CSF1, CXCL1, CXCL2, CXCL3, CXCL5, CXCL6, CXCL7, CXCL8, CXCL9, CXCL10, CXCL12, EGF or ICOSL; or

(8) The first binding domain targets and binds to TIGIT, and the second binding domain targets and binds to human TGF-β, HGF, EGF or VEGF; or

(9) The first binding domain targets and binds to CD155, and the second binding domain targets and binds to human TGF-β, VEGE HGF, EGF, CD47 or CD155, or

(10) The first binding domain targets and binds to Claudin 18.2, HER-2, mesothelin, BCMA, SSTR2, GPRC5D, PSMA, FCRH5, CD33, CD123, CD20, A33, CEA, CD28, DLL3, EGFR, VEGFR, VEGFR2, VEGF-A, Nectin-4, FGFR, C-met, RANKL, PDGF, PDGFR, PDGFα, DLL-4, Ang-1 or Ang-2, and the second binding domain targets and binds to human CTLA-4, TGF-β, VEGF, FGL1, LAG3, 4-1BB, OX40, CD27, CD28, CD47, CD155, HGF, CSF1, CXCL1, CXCL2, CXCL3, CXCL5, CXCL6, CXCL7, CXCL8, CXCL9, CXCL10, CXCL12, EGF or ICOSL.

Further, the first binding domain and the receptor or ligand are selected from the following combinations:

(I) The first binding domain targets and binds to PD-L1, and the receptor or ligand is selected from TGF-βRII, VEGFR, LAG-3, SIRPα, TIGIT, C-MET, CSF1R, CXCR2, CXCR3, CXCR4, EGFR or ICOS; or

(2) The first binding domain targets and binds to PD-1, and the receptor or ligand is selected from human CD80, CD86, VEGFR, EGFR, FGL1, CD70, 4-1BBL or OX40L, or

(3) The first binding domain targets and binds to CTLA-4, and the receptor or ligand is selected from human CD80, CD86, VEGFR, c-MET, EGFR, FULL CD70, 4-1BBL or OX40L; or

(4) The first binding domain targets and binds to LAG-3, and the receptor or ligand is selected from human VEGFR, c-MET EGFR, CD80, CD86, CD70, 4-1BBL or OX40L, or

(5) The first binding domain targets and binds to FGL1, and the receptor or ligand is selected from human TGF-βRII, VEGFR, SIRPα, c-MET, CSF1R, CXCR2, CXCR3, CXCR4, EGFR or ICOS; or

(6) The first binding domain targets and binds to TIM-3, and the receptor or ligand is selected from human VEGFR, c-MET, EGFR, FGL1, CD70, 4-1BBL or OX40L; or

(7) The first binding domain targets and binds to Galectin-9, and the receptor or ligand is selected from human TGF-βRII, VEGFR, SIRPα, c-MET, CSF1R, CXCR2, CXCR3, CXCR4, EGFR or ICOS; or

(8) The first binding domain targets and binds to TIGIT, and the receptor or ligand is selected from human TGF-βRII, c-MET, EGFR or VEGFR; or

(9) The first binding domain targets and binds to CD155, and the receptor or ligand is selected from human TGF-βRII, VEGFR, c-MET, EGFR, SIRPα, or TIGIT; or

(10) The first binding domain targets and binds to Claudin 18.2, HER-2, mesothelin, BCMA, SSTR2, GPRC5D, PSMA, FCRH5, CD33, CD123, CD20, A33, CEA, CD28, DLL3, EGFR, VEGFR, VEGFR2, Nectin-4, EGFR, c-MET, RANKL, PDGF, PDGFR, PDGFRα, DLL4 Ang-1 or Ang-2, and the receptor or ligand is selected from human CTLA-4, CD80, CD86, TGF-βRII, VEGFR, FGL1, LAG3, 4-1BB, OX40, CD27, CD28, CD70, c-MET, SIRPα, TIGIT, CSF1R, CXCR2, CXCR3, CXCR4, EGFR or ICOS.

Further, the fragment of the receptor or ligand comprises a fragment of the extracellular region and a fragment of the binding domain of the receptor or ligand.

Further, the first binding domain targets and binds to PD-L1; the second binding domain is a fragment of human TGF-βRII; or the first binding domain targets and binds to PD-1, and the second binding domain comprises human CD80 ECD, CD80 IgV region, human CD80 IgVIgC, VEGFR1 ECD, VEGFR2 ECD or a combination of the second extracellular region of VEGFR1 and the third extracellular region of VEGFR2; or the first binding domain targets and binds to Claudin 18.2, HER-2 or EGFR, and the second binding domain is selected from human CD80 ECD, CD80 IgV region, human CD80 IgVIgC, VEGFR1 ECD, VEGFR2 ECD or a combination of the second extracellular region of VEGFR1 and the third extracellular region of VEGFR2.

Further, the second binding domain is selected from:

(1) Human TGF-βRII comprising the sequence as shown in SEQ ID NO: 31, 131 or 132, or a sequence having an identity of greater than 80%, 85%, 90%, 95%, 96%, 97%, 98%, 99% compared with the sequence as shown in SEQ ID NO: 31, 131 or 132, or an amino acid sequence having 1, 2, 3, 4, 5, 6, 7, 8, 9, or 10 amino acid substitutions or deletions on the sequence as shown in SEQ ID NO: 31, 131 or 132; or

(2) CD80 ECD comprising the sequence as shown in SEQ ID NO: 32 or 133, or a sequence having an identity of greater than 80%, 85%, 90%, 95%, 96%, 97%, 98%, 99% compared with the sequence as shown in SEQ ID NO: 32 or 133, or an amino acid sequence having 1, 2, 3, 4, 5, 6, 7, 8, 9, or 10 amino acid substitutions or deletions on the sequence as shown in SEQ ID NO: 32 or 133; or

(3) A combination of the second extracellular region of VEGFR1 and the third extracellular region of VEGFR2, comprising the sequence as shown in SEQ ID NO: 33, or a sequence having an identity of greater than 80%, 85%, 90%, 95%, 96%, 97%, 98%, 99% compared with the sequence as shown in SEQ ID NO: 33, or an amino acid sequence having 1, 2, 3, 4, 5, 6, 7, 8, 9, or 10 amino acid substitutions or deletions on the sequence as shown in SEQ ID NO: 33.

Further, the C-terminus or/and N-terminus of the second binding domain has a peptide linker, and the peptide linker consists of 2-30 amino acids.

Further, the peptide linker may be:

(1) (GGGGS)n;
or
(SEQ ID NO: 80)
(2) AKTTPKLEEGEFSEAR;
or
(SEQ ID NO: 81)
(3) AKTTPKLEEGEFSEARV;
or
(SEQ ID NO: 82)
(4) AKTTPKLGG;
or
(SEQ ID NO: 83)
(5) SAKTTPKLGG;
or
(SEQ ID NO: 84)
(6) SAKTTP;
or
(SEQ ID NO: 85)
(7) RADAAP;
or
(SEQ ID NO: 86)
(8) RADAAPTVS;
or
(SEQ ID NO: 87)
(9) RADAAAAGGPGS;
or
(SEQ ID NO: 88)
(10) RADAAAA;
or
(SEQ ID NO: 89)
(11) SAKTTPKLEEGEFSEARV;
or
(SEQ ID NO: 90)
(12) ADAAP;
or
(SEQ ID NO: 91)
(13) DAAPTVSIFPP;
or
(SEQ ID NO: 92)
(14) TVAAP;
or
(SEQ ID NO: 93)
(15) TVAAPSVFIFPP;
or
(SEQ ID NO: 94)
(16) QPKAAP;
or
(SEQ ID NO: 95)
(17) QPKAAPSAVTLFPP;
or
(SEQ ID NO: 96)
(18) AKTTPP;
or
(SEQ ID NO: 97)
(19) AKTTPPSVTPLAP;
or
(SEQ ED NO: 98)
(20) AKTTAP;
or
(SEQ ID NO: 99)
(21) AKTTAPSVYPLAP;
or
(SEQ ID NO: 100)
(22) ASTKGP;
or
(SEQ ID NO: 101)
(23) ASTKGPSVFPLAP;
or
(SEQ ID NO: 102)
(24) GENKVEYAPALMALS;
or
(SEQ ID NO: 103)
(25) GPAKELTPLKEAKVS;
or
(SEQ ID NO: 104)
(26) GHEAAAVMQVQYPAS;
or
(SEQ ID NO: 105)
(27) GGGGSGGGGSGGGGSA,

wherein n is equal to 1, 2, 3 or 4;

when n=1, the (GGGGS)n is GGGGS (SEQ ID NO: 120);

when n=2, the (GGGGS)n is GGGGSGGGGS (SEQ ID NO: 121) or (GGGGS)₂;

when n=3, the (GGGGS)n is GGGGSGGGGSGGGGS (SEQ ID NO: 122) or (GGGGS)₃;

when n=4, the (GGGGS)n is GGGGSGGGGSGGGGSGGGGS (SEQ ID NO: 123) or (GGGGS)₄.

Further, the size of the inserted second binding domain does not exceed 235, 240, 250, 260, 270, 280, 290 or 300 amino acids.

Further, the insertion site of the second binding domain is located in a middle front part of the hinge region, and the insertion site does not affect the formation of disulfide bonds of the immunoglobulin, wherein the middle front part of the hinge region generally refers to the part before 231A. Further, part of the amino acids in the hinge region before and after the insertion site are substituted or deleted. For example, the hinge region contains D221G and/or C220V mutations.

Further, the IgG is selected from mammalian IgG, humanized IgG, and human IgG, and the mammal includes mouse and rat; preferably, the IgG is IgG1, IgG2, IgG3 or IgG4.

Further, the Fc region of the fusion protein is a lycosylated or deglycosylated, or has reduced fucosylation or is afucosylated.

Further, the IgG is Atezolizumab, Avelumab, Durvalumab, Nivolumab, Pembrolizumab, Cemiplimab or Ipilimumab. Preferably, the IgG is Atezolizumab, the amino acid sequence of the heavy chain of the IgG is shown in SEQ ID NO: 106, and the amino acid sequence of the light chain of the IgG is shown in SEQ ID NO: 107; or the IgG is Avelumab, the amino acid sequence of the heavy chain of the IgG is shown in SEQ ID NO: 108, and the amino acid sequence of the light chain of the IgG is shown in SEQ ID NO: 109; or the IgG is Durvalumab, the amino acid sequence of the heavy chain of the IgG is shown in SEQ ID NO: 110, and the amino acid sequence of the light chain of the IgG is shown in SEQ ID NO: 111; or the IgG is Nivolumab, the amino acid sequence of the heavy chain of the IgG is shown in SEQ ID NO: 112, and the amino acid sequence of the light chain of the IgG is shown in SEQ ID NO: 113; or the IgG is Pembrolizumab, the amino acid sequence of the heavy chain of the IgG is shown in SEQ ID NO: 114, and the amino acid sequence of the light chain of the IgG is shown in SEQ ID NO: 115; or the IgG is Ipilimumab, the amino acid sequence of the heavy chain of the IgG is shown in SEQ ID NO: 116, and the amino acid sequence of the light chain of the IgG is shown in SEQ ID NO: 117; or the IgG is Cemiplimab, the amino acid sequence of the heavy chain of the IgG is shown in SEQ ID NO: 118, and the amino acid sequence of the light chain of the IgG is shown in SEQ ID NO: 119.

Specifically, the present disclosure provides a Hibody structure fusion protein with a first binding domain targeting human PD-L1, the CDRs of the heavy chain variable region and/or the CDRs of the light chain variable region in the Fab of the IgG have the same CDR sequence as the antibody defined by the following sequence, or has 1-2 amino acid substitutions on e CDRs of the antibody defined by the following sequence, the antibody is defined as follows:

(1) The amino acid sequence of the heavy chain variable region is shown in SEQ ID NO: 66; and/or

(2) The amino acid sequence of the light chain variable region is shown in SEQ ID NO: 67.

Further, the CDRs of the heavy chain variable region and/or the CDRs of the light chain variable region in the Fab of the IgG are as follows:

(1) For the heavy chain variable region, the amino acid sequence of CDR1 is selected from SEQ CIO NOs: 1-5 and amino acid sequences having 1 or 2 amino acid substitutions on SEQ NOs: 1-5; the amino acid sequence of CDR2 is selected from SEQ ID NOs: 6-10 and amino acid sequences having 1 or 2 amino acid substitutions on SEQ ID NOs: 6-10; the amino acid sequence of CDR3 is selected from SEQ ID NOs: 11-15 and amino acid sequences having 1 or 2 amino acid substitutions on SEQ ID NOs: 11-15; and/or

(2) For the light chain variable region, the amino acid sequence of CDR1 is selected from SEQ ID NOs: 16-20 and amino acid sequences having 1 or 2 amino acid substitutions on SEQ ID NOs: 16-20; the amino acid sequence of CDR2 is selected from SEQ ID NOs: 21-25 and amino acid sequences having 1 or 2 amino acid substitutions on SEQ ID NOs: 21-25; the amino acid sequence of CDR3 is selected from SEQ ID NOs: 26-30 and amino acid sequences having 1 or 2 amino acid substitutions on SEQ ID NOs: 26-30.

In a particular embodiment, according to different measurement methods or system identifications used, the complementarity determining regions CDRs 1-3 of the corresponding heavy chain and light chain variable regions are shown in Table 2.

TABLE 2
Amino acid sequences of heavy chain and light chain variable region CDRs 1-3
of the anti-PD-L1 antibody

| Category | System | CDR1 | CDR2 | CDR3 |
|---|---|---|---|---|
| Heavy | Chothia | SEQ ID NO: 1 | SEQ ID NO: 6 | SEQ ID NO: 11 |
| chain | | GESLSRY | WGVGT | NWGTADYFDY |
| | AbM | SEQ ID NO: 2 | SEQ ID NO: 7 | SEQ ID NO: 12 |
| | | GFSLSRYSVH | MIWGVGTTD | NWGTADYFDY |
| | Kabat | SEQ ID NO: 3 | SEQ ID NO: 8 | SEQ ID NO: 13 |
| | | RYSVH | MIWGVGTTDYNSALKS | NWGTADYFDY |
| | Contact | SEQ ID NO: 4 | SEQ ID NO: 9 | SEQ ID NO: 14 |
| | | SRYSVH | WLGMIWGVGTTD | ARNWGTADYFD |
| | IMGT | SEQ ID NO: 5 | SEQ ID NO: 10 | SEQ ID NO: 15 |
| | | GFSLSRYS | IWGVGTT | ARNWGTADYFDY |
| Light | Chothia | SEQ ID NO: 16 | SEQ ID NO: 21 | SEQ ID NO: 26 |
| chain | | RASKSVHTSGYSYMH | LASNLES | QHSGELPYT |
| | AbM | SEQ ID NO: 17 | SEQ ID NO: 22 | SEQ ID NO: 27 |
| | | RASKSVHTSGYSYMH | LASNLES | QHSGELPYT |
| | Kabat | SEQ ID NO: 18 | SEQ ID NO: 23 | SEQ ID NO: 28 |
| | | RASKSVHTSGYSYMH | LASNLES | QHSGELPYT |
| | Contact | SEQ ID NO: 19 | SEQ ID NO: 24 | SEQ ID NO: 29 |
| | | HTSGYSYMHWY | LLIYLASNLE | QHSGELPY |
| | IMGT | SEQ ID NO: 20 | SEQ ID NO: 25 | SEQ ID NO: 30 |
| | | KSVHTSGYSY | LAS | QHSGELPYT |

Further, the fusion protein comprises the following CDR sequences,

(1) The amino acid sequences of CDRs 1-3 of the heavy chain variable region are SEQ ID NOs: 1, 6, 11, or an amino acid sequence having 1 or 2 amino acid substitutions on SEQ ID NOs: 1, 6, 11; and/or an amino acid sequence of CDRs 1-3 of the light chain variable region are SEQ ID NOs: 16, 21, 26, or an amino acid sequence having 1 or 2 amino acid substitutions on SEQ ID NOs: 16, 21, 26; or

(2) The amino acid sequences of CDRs 1-3 of the heavy chain variable region are SEQ ID NOs: 2, 7, 12, or an amino acid sequence having 1 or 2 amino acid substitutions on SEQ ID NOs: 2, 7, 12; and/or an amino acid sequence of CDRs 1-3 of the light chain variable region are SEQ ID NOs: 17, 22, 27, or an amino acid sequence having 1 or 2 amino acid substitutions on SEQ ID NOs: 17, 22, 27; or

(3) The amino acid sequences of CDRs 1-3 of the heavy chain variable region are SEQ ID NOs: 3, 8, 13, or an amino acid sequence having 1 or 2 amino acid substitutions on SEQ ID NOs: 3, 8, 13; and/or an amino acid sequence of CDRs 1-3 of the light chain variable region are SEQ ID NOs: 18, 23, 28, or an amino acid sequence having 1 or 2 amino acid substitutions on SEQ ID NOs: 18, 23, 28; or

(4) The amino acid sequences of CDRs 1-3 of the heavy chain variable region are SEQ ID NOs: 4, 9, 14, or an amino acid sequence having 1 or 2 amino acid substitutions on SEQ ID NOs: 4, 9, 14; and/or an amino acid sequence of CDRs 1-3 of the light chain variable region are SEQ ID NOs: 19, 24, 29, or an amino acid sequence having 1 or 2 amino acid substitutions on SEQ ID NOs: 19, 24, 29; or

(5) The amino acid sequences of CDRs 1-3 of the heavy chain variable region are SEQ ID NOs: 5, 10, 15, or an amino acid sequence having 1 or 2 amino acid substitutions on SEQ ID NOs: 5, 10, 15; and/or an amino acid sequence of CDRs 1-3 of the light chain variable region are SEQ ID NOs: 20, 25, 30, or an amino acid sequence having 1 or 2 amino acid substitutions on SEQ ID NOs: 20, 25, 30.

Further, in the heavy chain of the fusion protein, the amino acid sequences of CDRs 1-3 of the heavy chain variable region are SEQ ID NOs: 3, 8, 13; and/or an amino acid sequence of CDRs 1-3 of the light chain variable region are SEQ ID NOs: 18, 23, 28.

Further, the sequences of the heavy chain variable region and the light chain variable region of the fusion protein are as follows,

(1) The heavy chain variable region has a sequence as shown in SEQ ID NO: 66, or a sequence that has the same CDRs 1-3 as SEQ ID NO: 66 and has an identity of greater than 80%, 85%, 90%, 95%, 96%, 97%, 98%, 99% compared with SEQ ID NO: 66; and/or

(2) The light chain variable region has a sequence as shown in SEQ ID NO: 67, or a sequence that has the same CDRs 1-3 as SEQ ID NO: 67 and has an identity of greater than 80%, 85%, 90%, 95%, 96%, 97%, 98%, 99% compared with SEQ ID NO: 67.

More specifically, the amino acid sequences of the heavy chain and light chain of the fusion protein are as follows,

(1) The heavy chain of the fusion protein has an amino acid sequence as shown in SEQ ID NO: 72, or a sequence having an identity of greater than 80%, 85%, 90%, 95%, 96%, 97%, 98%, 99% compared with SEQ ID NO: 72;

(2) The light chain of the fusion protein has an amino acid sequence as shown in SEQ ID NO: 73, or a sequence having an identity of greater than 80%, 85%, 90%, 95%, 96%, 97%, 98%, 99% compared with SEQ ID NO: 73.

Further, the amino acid sequences of the heavy chain and light chain of the fusion protein are as follows,

(1) The amino acid sequence of the heavy chain of the fusion protein has 1-15 amino acid site mutations or has an alternative peptide linker compared with SEQ ID NO: 72;

(2) The amino acid sequence of the light chain of the fusion protein has 1-10 amino acid site mutations compared with SEQ ID NO: 73.

Specifically, the present disclosure provides a Hibody structure fusion protein with a first binding domain targeting human PD-1, wherein the CDRs of the heavy chain variable region and/or the CDRs of the light chain variable region in the Fab have the same CDR sequence as the antibody defined by the following sequence, or has 1-2 amino acid substitutions on the CDRs of the antibody defined by the following sequence, the antibody is defined as follows:

(1) The amino acid sequence of the heavy chain variable region is shown in SEQ ID NO: 64; and/or

(2) The amino acid sequence of the light chain variable region is shown in SEQ ID NO: 65.

Further, the CDRs of the heavy chain variable region and/or the CDRs of the light chain variable region in the Fab in the fusion protein are as follows:

(1) For the heavy chain variable region, the amino acid sequence of CDR1 is selected from SEQ ID NOs: 34-38 and amino acid sequences having 1 or 2 amino acid substitutions on SEQ ID NOs: 34-38; the amino acid sequence of CDR2 is selected from SEQ ID NOs: 39-43 and amino acid sequences having 1 or 2 amino acid substitutions on SEQ ID NOs: 39-43; the amino acid sequence of CDR3 is selected from SEQ ID NOs: 44-48 and amino acid sequences having 1 or 2 amino acid substitutions on SEQ ID NOs: 44-48; and/or

(2) For the light chain variable region, the amino acid sequence of CDR1 is selected from SEQ ID NOs: 49-53 and amino acid sequences having 1 or 2 amino acid substitutions on SEQ ID NOs: 49-53; the amino acid sequence of CDR2 is selected from SEQ NOs: 54-58 and amino acid sequences having 1 or 2 amino acid substitutions on SEQ ID NOs: 54-58; the amino acid sequence of CDR3 is selected from SEQ ID NOs: 59-63 and amino acid sequences having 1 or 2 amino acid substitutions on SEQ ID NOs: 59-63

In a particular embodiment, according to different measurement methods or system identifications used, the complementarity determining regions CDRs 1-3 of the corresponding heavy chain and light chain variable regions are shown in Table 3.

TABLE 3
Amino acid sequences of heavy chain and light chain variable region CDRs 1-3
of the anti-PD4 antibody

| Category | System | CDR1 | CDR2 | CDR3 |
|---|---|---|---|---|
| Heavy | Chothia | SEQ ID NO: 34 | SEQ ID NO: 39 | SEQ ID NO: 44 |
| chain | | GITFSNS | WYDGSK | NDDY |
| | AbM | SEQ ID NO: 35 | SEQ ID NO: 40 | SEQ ID NO: 45 |
| | | GITFSNSGMH | VIWYDGSKRY | NDDY |
| | Kabat | SEQ ID NO: 36 | SEQ ID NO: 41 | SEQ ID NO: 46 |
| | | NSGMH | VIWYDGSKRYYADSVKG | NDDY |
| | Contact | SEQ ID NO: 37 | SEQ ID NO: 42 | SEQ ID NO: 47 |
| | | SNSGMH | WVAVIWYDGSKRY | ATNDD |
| | IMGT | SEQ ID NO: 38 | SEQ ID NO: 43 | SEQ ID NO: 48 |
| | | GITFSNSG | IWYDGSKR | ATNDDY |
| Light | Chothia | SEQ ID NO: 49 | SEQ ID NO: 54 | SEQ. ID NO: 59 |
| chain | | RASQSVSSYLA | DASNRAT | QQSSNWPRT |
| | AbM | SEQ ID NO: 50 | SEQ ID NO: 55 | SEQ ID NO: 60 |
| | | RASQSVSSYLA | DASNRAT | QQSSNWPRT |
| | Kabat | SEQ ID NO: 51 | SEQ ID NO: 56 | SEQ ID NO: 61 |
| | | RASQSVSSYLA | DASNRAT | QQSSNWPRT |
| | Contact | SEQ ID NO: 52 | SEQ ID NO: 57 | SEQ ID NO: 62 |
| | | SSYLAWY | LLIYDASNRA | QQSSNWPR |
| | IMGT | SEQ ID NO: 53 | SEQ ID NO: 58 | SEQ ID NO: 63 |
| | | QSVSSY | DAS | QQSSNWPRT |

Further, the CDRs 1-3 of the variable region of the fusion protein is defined as follows,

(1) The amino acid sequences of CDRs 1-3 of the heavy chain variable region are SEQ ID NOs: 34, 39, 44, or an amino acid sequence having 1 or 2 amino acid substitutions on SEQ ID NOs: 34, 39, 44; and/or an amino acid sequence of CDRs 1-3 of the light chain variable region are SEQ ID NOs: 49, 54, 59, or an amino acid sequence having 1 or 2 amino acid substitutions on SEQ ID NOs: 49, 54, 59; or

(2) The amino acid sequences of CDRs 1-3 of the heavy chain variable region are SEQ ID NOs: 35, 40, 45, or an amino acid sequence having 1 or 2 amino acid substitutions on SEQ ID NOs: 35, 40, 45; and/or an amino acid sequence of CDRs 1-3 of the light chain variable region are SEQ ID NOs: 50, 55, 60, or an amino acid sequence having 1 or 2 amino acid substitutions on SEQ ID NOs: 50, 55, 60; or

(3) The amino acid sequences of CDRs 1-3 of the heavy chain variable region are SEQ ID NOs: 36, 41, 46, or an amino acid sequence having 1 or 2 amino acid substitutions on SEQ ID NOs: 36, 41, 46; and/or an amino acid sequence of CDRs 1-3 of the light chain variable region are SEQ ID NOs: 51, 56, 61, or an amino acid sequence having 1 or 2 amino acid substitutions on SEQ ID NOs: 51, 56, 61; or

(4) The amino acid sequences of CDRs 1-3 of the heavy chain variable region are SEQ ID NOs: 37, 42, 47, or an amino acid sequence having 1 or 2 amino acid substitutions on SEQ ID NOs: 37, 42, 47; and/or an amino acid sequence of CDRs 1-3 of the light chain variable region are SEQ ID NOs: 52, 57, 62, or an amino acid sequence having 1 or 2 amino acid substitutions on SEQ ID NOs: 52, 57, 62; or

(5) The amino acid sequences of CDRs 1-3 of the heavy chain variable region are SEQ ID NOs: 38, 43, 48, or an amino acid sequence having 1 or 2 amino acid substitutions on SEQ ID NOs: 38, 43, 48; and/or an amino acid sequence of CDRs 1-3 of the light chain variable region are SEQ ID NOs: 53, 58, 63, or an amino acid sequence having 1 or 2 amino acid substitutions on SEQ ID NOs: 53, 58, 63.

Further, the CDRs 1-3 of the variable region of the fusion protein is defined as follows: the amino acid sequences of CDRs 1-3 of the heavy chain variable region are SEQ ID NOs: 36, 41, 46; and/or an amino acid sequence of CDRs 1-3 of the light chain variable region are SEQ ID NOs: 51, 56, 61.

Further, the amino acid sequences of the heavy chain variable region and the light chain variable region of the fusion protein are as follows,

(1) The heavy chain variable region has a sequence as shown in SEQ ID NO: 64, or a sequence that has the same CDRs 1-3 as SEQ ID NO: 64 and has an identity of greater than 80%, 85%, 90%, 95%, 96%, 97%, 98%, 99% compared with SEQ ID NO: 64; and/or

(2) The light chain variable region has a sequence as shown in SEQ ID NO: 65, or a sequence that has the same CDRs 1-3 as SEQ ID NO: 65 and has an identity of greater than 80%, 85%, 90%, 95%, 96%, 97%, 98%, 99% compared with SEQ ID NO: 65.

More specifically, the amino acid sequences of the heavy chain and light chain of the fusion protein are defined as follows:

(1) The heavy chain of the fusion protein has an amino acid sequence as shown in SEQ ID NO: 68, or a sequence having an identity of greater than 80%, 85%, 90%, 95%, 96%, 97%, 98%, 99% compared with SEQ ID NO: 68;

(2) The light chain of the fusion protein has an amino acid sequence as shown in SEQ ID NO: 69, or a sequence having an identity of greater than 80%, 85%, 90%, 95%, 96%, 97%, 98%, 99% compared with SEQ ID NO: 69.

Further, the amino acid sequences of the heavy chain and light chain of the fusion protein are defined as follows:

(1) The amino acid sequence of the heavy chain of the fusion protein has 1-15 amino acid site mutations or has an alternative peptide linker compared with SEQ ID NO: 68;

(2) The amino acid sequence of the light chain of the fusion protein has 1-10 amino acid site mutations compared with SEQ ID NO: 69.

More specifically, the amino acid sequences of the heavy chain and light chain of the fusion protein are defined as follows:

(1) The heavy chain of the fusion protein has an amino acid sequence as shown in SEQ ID NO: 70, or a sequence having an identity of greater than 80%, 85%, 90%, 95%, 96%, 97%, 98%, 99% compared with SEQ ID NO: 70;

(2) The light chain of the fusion protein has an amino acid sequence as shown in SEQ ID NO: 71, or a sequence having an identity of greater than 80%, 85%, 90%, 95%, 96%, 97%, 98%, 99% compared with SEQ ID NO: 71.

Further, the amino acid sequences of the heavy chain and light chain of the fusion protein are defined as follows:

(1) The amino acid sequence of the heavy chain of the fusion protein has 1-15 amino acid site mutations or has an alternative peptide linker compared with SEQ ID NO: 70;

(2) The amino acid sequence of the light chain of the fusion protein has 1-10 amino acid site mutations compared with SEQ ID NO: 71.

The present disclosure also provides an isolated polynucleotide encoding the above fusion protein.

The present disclosure also provides a nucleic acid construct comprising the above polynucleotide, preferably the nucleic acid construct is a vector.

The present disclosure also provides a host cell comprising the above polynucleotide or the above nucleic acid construct or the vector, preferably the cell is a prokaryotic cell, eukaryotic cell, yeast cell, mammal cell, E. coli cell or CHO cell, NS0 cell, Sp2/0 cell, or BHK cell.

The present disclosure also provides a pharmaceutical composition comprising the above fusion protein and a pharmaceutically acceptable carrier.

The present disclosure also provides a method for treating tumor or cancer, comprising the step of administering the above fusion protein or the pharmaceutical composition to a subject in need of a treatment or relief.

The present disclosure also provides use of the above fusion protein, the polynucleotide, the nucleic acid construct or the vector, or the pharmaceutical composition in the manufacture of a medicament for the treatment or prevention of a tumor or cancer. The tumor or cancer includes a solid tumor or a non-solid tumor.

The present disclosure also provides a diagnostic kit comprising the above fusion protein.

The present disclosure also provides a method of manufacturing the fusion protein, comprising culturing the host cell under conditions allowing the expression of the above nucleic acid construct, and recovering the produced fusion protein from the culture.

The Hibody structure fusion protein provided in the present disclosure has the same advantages of expression and production as IgG because the second binding domain is inserted in the hinge region, and can be seamlessly applicable to the existing IgG expression platform and purification platform, which greatly reduces the subsequent development cost; compared with the bispecific antibody constructed by the existing bispecific antibody platform, the bispecific antibody constructed by the Hibody platform has a higher expression level and does not have the problem of light and heavy chain mismatches, which also greatly reduces the cost of antibody production. In addition, it is surprising to find that inserting a second binding domain of a certain size in the two hinge regions not only does not affect the binding activity of the Fab region of the fusion protein and the stability of the protein, but also further improves the stability and obtains a longer half-life. More notably, the binding activity of the second binding domain to the target binding site is significantly improved compared to that of the corresponding soluble natural binding fragment monomer to the corresponding target, which may lie in that since the receptor or ligand of the selected second binding domain will form a dimerization or multimerization structure in the natural signaling pathway to activate or inhibit the signaling pathway, when the second binding domain is inserted into the hinge region, its spatial positional relationship in the hinge region promotes the formation of the dimerization of the corresponding receptor or ligand or its fragment, so that by imitating the natural signal pathway, a more significantly improved binding activity is generated, which makes the fusion protein have a greatly increased activation or inhibition ability compared with a soluble receptor or ligand or its fragment. Besides, through a reasonable combination of the two binding domains, the target of receptor inhibition or receptor activation can be flexibly selected to achieve an effective synergistic effect. The bispecific fusion protein provided in the present disclosure has a more significant disease treatment effect to effectively treat or control the disease process, especially in various tumors and cancers, compared with the separate administration of two target drugs or the bispecific, antibody of the prior art.

### BRIEF DESCRIPTION OF DRAWINGS

FIG. 1 Schematic diagram of Hibody structure fusion protein.

FIG. 2 shows the inhibition curve of Hib-PDV and its control group on mouse colon cancer model tumor.

FIG. 3 shows the inhibition curve of Hib-PDC and its control group on mouse colon cancer tumor.

FIG. 4 is a comparison chart of Hib-PLT and its control group on promoting cell secretion of IFN-γ factor at the level of 10-100 nM.

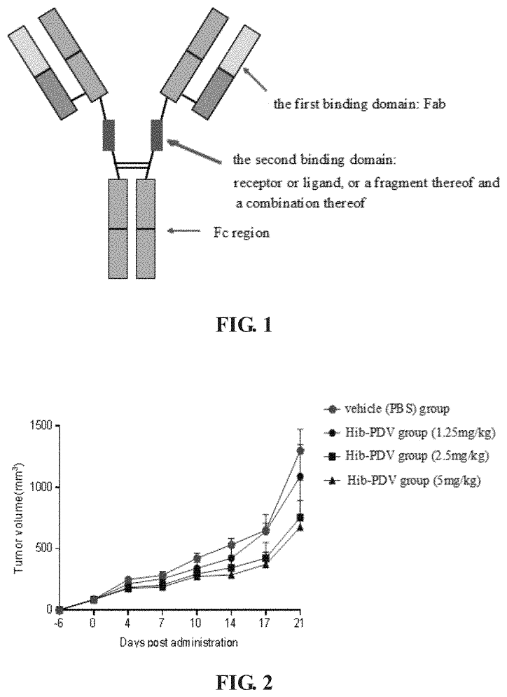

### DETAILED DESCRIPTION

### Definitions

Unless otherwise defined, all technical and scientific terms used herein have the same meaning as commonly understood by those of ordinary skill in the art related to the present disclosure. Those skilled in the art can refer to the Dictionary of Cell and Molecular Biology, third edition, 1999, Academic Press; the Concise Dictionary of Biomedicine and Molecular Biology, Juo, Pei-Show, second edition, 2002, CRC Press; and the Oxford Dictionary of Biochemistry and Molecular Biology, revised edition, 2000, Oxford University Press.

The amino acids involved in the present disclosure can be represented by their commonly known three-letter symbols or by the single-letter symbols recommended by the IUPAC-IUB Biochemical Nomenclature Commission. Likewise, nucleotides can be represented by their generally recognized one-letter codes.

In the present disclosure, the determination or numbering method of the complementarity determining region (CDR) of the antibody variable domain includes IMGT, Kabat (such as stated in Kabat et al., Sequences of Proteins of Immunological Interest, 5th edition, Public Health Service, National Institutes of Health, Bethesda Md. (1991)), Chothia, AbM and Contact method.

For the purpose of the present invention, “identity”, “consistency” or “similarity” between two nucleic acid or amino acid sequences refers to the percentage of the same nucleotides or amino acid residues between the two sequences to be compared Obtained after the optimal alignment. The percentage is purely statistical and the differences between the two sequences are randomly distributed and cover their full length. Comparisons between two nucleic acid or amino acid sequences are usually performed by comparing these sequences after aligning them in an optimal manner, and can be performed over a segment or over a “comparison window”. In addition to manual implementation, the optimal alignment for comparing sequences can also performed through the local homology algorithm of Smith and Waterman (1981) [Ad. App. Math. 2: 482], through the local homology algorithm of Neddleman and Wunsch (1970) [J. Mot. Biol, 48: 443], through the similarity search method of Pearson and Lipman (1988) [Proc. Natl. Acad, Sci. USA 85: 2444), and through the computer software using these algorithms (GAP, BESTFIT, FASTA and TFASTA in the Wisconsin Genetics Software Package, Genetics Computer Group, 575 Science Dr., Madison, Wis., or through BLAST N or BLAST P comparison software).

As broadly defined, the immunoglobulin involved in the present disclosure refers to an animal protein with antibody activity, consisting of two identical light chains and two identical heavy chains. It is an important class of immune effector molecules and a protein produced by lymphocytes of higher animal immune system, and can be transformed into antibodies by induction of antigens. Due to different structures, it can be divided into 5 types: IgG, IgA, IgM, IgD and IgE. Preferably, the immunoglobulin involved in the present disclosure is IgG.

As broadly defined, the receptor involved in the present disclosure is a type of special protein that exists in the cell membrane or in the cell and can bind to a specific signal molecule outside the cell to activate a series of biochemical reactions in the cell to cause the cell to produce a corresponding effect under external stimuli. The biologically active substances that bind to receptors are collectively referred to as ligands. The binding of the receptor and the ligand results in a molecular conformation change, which causes cellular responses, such as mediating intercellular signal transduction, intercellular adhesion, endocytosis, and the like.

The bispecific fusion protein involved in the present disclosure is based on the structure of an immunoglobulin with a second binding domain inserted in its hinge region. Therefore, the bispecific fusion protein involved in the present disclosure has two binding domains.

### EXAMPLES

The present disclosure will be further illustrated by way of the following examples. It should be noted that the following examples are for further elaboration and explanation of the present disclosure, and should not be regarded as limiting the present disclosure.

### Example 1 Construction of Proteins

in this example, the following protein molecules were constructed using conventional methods in the art, and expressed transiently or stably according to conventional methods in the art:
(1) Three Hibody bispecific fusion proteins: Hib-PLT, Hib-PDC, and Hib-PDV;
(2) Control proteins 1-3: constructed according to the bifunctional molecular structure in WO2015/118175;
(3) Other control proteins: TGF-β-R-His, VEGF-R-His, CD80-His, PD-L1 control antibodies.

TABLE 4
Target combinations of fusion protein

| Fusion protein | First binding | Second binding | Second binding domain fragment | Peptide |
|---|---|---|---|---|
| code | domain target | domain target | inserted | linker |
| Hib-PLT | PD-L1 | TGF-β | TGF-βRII extracellular region fragment | (GGGGS)₃ |
| Hib-PDC | PD-1 | CD28 | CD80 | (GGGGS)₃ |
| Hib-PDV | PD-1 | VEGF | The combination of the second | (GGGGS)₃ |
| | | | extracellular region of VEGFR1 and the | |
| | | | third extracellular region of VEGFR2 | |
| Control protein 1 | PD-L1 | TGF-β | TGF-βRII extracellular region fragment | (GGGGS)₃ |
| Control protein 2 | PD-1 | CD28 | CD80 | (GGGGS)₃ |
| Control protein 3 | PD-1 | VEGF | The combination of the second | (GGGGS)₃ |
| | | | extracellular region of VEGFR1 and the | |
| | | | third extracellular region of VEGFR2 | |

The nucleic acid sequences and amino acid sequences of the heavy chain and light chain of the fusion protein Hib-PLT are as follows:

Nucleic acid sequence of fusion protein Hib-PLT heavy chain (SEQ ID NO: 78):

CAGGTGCAGCTGCAGGAGTCCGGACCAGGACTGGTGAAGCCATCCGAGACCCTGAGCCTGAC
CTGTACAGTGTCCGGCTTCAGCCTGTCTAGGTACAGCGTGCACTGGATCAGACAGCCACCTGG
CAAGGGACTGGAGTGGCTGGGCATGATCTGGGGCGTGGGCACCACAGACTACAACTCTGCTC
TGAAGTCCAGACTGACCATCAGCAAGGATACATCTAAGAATCAGTTCAGCCTGAAGCTGTCCA
GCGTGACCGCCGCTGACACAGCCGTGTACTACTGCGCTCGCAACTGGGGCACCGCCGACTACT
TCGACTATTGGGGCCAGGGCACCACAGTGACAGTGTCTTCCGCTAGCACCAAGGGCCCATCG
GTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCGTGGTGA
CCGTGCCCTCCAGCAGCTTGGGCACCCAGACCTACATCTGCAACGTGAATCACAAGCCCAGC
AACACCAAGGTGGACAAGAAAGTTGAGCCCAAATCTTGTGACAAAGGCGGGGGAGGCAGCG
GCGGCGGAGGAAGCGGGGGCGGAGGTAGCACCATCCCTCCACACGTGCAGAAGAGCGTG
AACAATGACATGATCGTCACCGACAACAACGGCGCCGTCAAGTTCCCCCAGCTGTGCAA
ATTCTGCGACGTGAGGTTCTCCACGTGCGACAACCAGAAGAGCTGTATGAGCAACTGCA
GCATCACATCCATCTGCGAAAAACCCCAGGAAGTGTGCGTCGCCGTCTGGCGGAAGAA
CGACGAGAACATCACACTGGAGACCGTGTGCCACGACCCCAAACTGCCCTACCACGACT
TCATCCTGGAGGACGCCGCCAGCCCAAAGTGCATCATGAAAGAGAAGAAGAAGCCGGG
CGAGACTTTCTTCATGTGCTCCTGCAGCTCCGACGAGTGCAACGATAATATCATCTTCAG
CGAAGAATACAACACATCTAACCCAGACGGAGGGGGCGGATCCGGGGGCGGCGGAAGCGG
CGGGGGGGGCAGCACTCACACNMCCCACCGTGCCCAGCACCTGAACTCCTGGGGGGACCGT
CAGTCTTCCTCTTCCCCCCAAAACCCAAGGACACCCTCATGATCTCCCGGACCCCTGAGGTCA
CATGCGTGGTGGTGGACGTGAGCCACGAAGACCCTGAGGTCAAGTTCAACTGGTACGTGGAC
GGCGTGGAGGTGCATAArGCCAAGACAAAGCCGCGGGAGGAGCAGIACAACAGCACGTACC
GTGTGGTCAGCGTCCTCACCGTCCTGCACCAGGACTGGCTGAAIGGCAAGGAGTACAAGTGC
AAGGTCTCCAACAAAGCCCTCCCAGCCCCCATCGAGAAAACCATCTCCAAAGCCAAAGGGCA
GCCCCGAGAACCACAGGIGTACACCCTGCCCCCATCCCGGGAGGAGArGACCAAGAACCAGG
TCAGCCTGACCTGCCTGGTCAAAGGCTICTAFCCCAGCGACATCGCCGTGGAGTGGGAGAGC
AAIGGGCAGCCGGAGAACAACTACAAGACCACGCCTCCCGTGCTGGACTCCGACGGCTCCTT
CTTCCTCTACAGCAAGCTCACCGTGGACAAGAGCAGGTGGCAGCAGGGGAACGTCTTCTCAT
GCTCCGTGAFGCNFGAGGCTCTGCACAACCACTACACGCAGAAGAGCCTCTCCCTGTCTCCGG
GTAANTGA

Note: The underlined and bold sequence is the TGF-βRII nucleic acid sequence.

Amino acid sequence of fusion protein Hib-PLT heavy chain (SEQ ID NO: 72):

| | |
|---|---|
| QVQLQESGPG LVKPSETLSL TCTVSGFSLS RYSVHWIRQP PGKGLEWLGM IWGVGITDYN | 60 |
| SALKSRLTIS KDTSKNQFSL KLSSVTAADT AVYYCARNWG TADYFDYWGQ GTTVTVSSAS | 120 |
| TKGPSVFPLA PSSKSTSGGT AALGCLVKDY FPEPVTVSWN SGALTSGVHT FPAVLQSSGL | 180 |
| 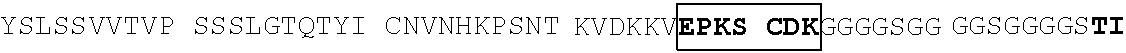 | 240 |
| PPHVQKSVNN DMIVTDNNGA VKFPQLCKFC DVRFSTCDNQ KSCMSNCSIT SICEKPQEVC | 300 |
| VAVWRKNDEN ITLETVCHDP KLPYHDFILE DAASPKCIMK EKKKPGETFF MCSCSSDECN | 360 |
| 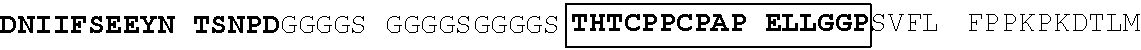 | 420 |
| ISRTPEVTCV VVDVSHEDPE VKFNWYVDGV EVHNAKTKPR EEQYNSTYRV VSVLTVLHQD | 480 |
| WLNGKEYKCK VSNKALPAPI EKTISKAKGQ PREPQVYTLP PSREEMTENQ VSLTCLVKF | 540 |
| YPSDIAVEWE SNGQPENNYK TTPPVLDSDG SFFLYSKLTV DKSRWQQGNV FSCSVMHEAL | 600 |
| HNHYTQKSLS LSPGK | 615 |

Note: The bold and framed fragment is the hinge region sequence, the peptide linker is in italics, and the underlined and bold sequence is the TGF-βRII amino acid sequence.

Nucleic acid sequence of fusion protein Hib-PLT light chain (SEQ ID NO: 79):

GATATCGTGCTGACCCAGTCTCCAGCTTCCCTGGCCGTGTCCCCAGGACA
GAGGGCCACCATCACATGTCGGGCTTCCAAGAGCGTGCACACAAGCGGCT
ACTCTTATATGCATTGGTACCAGCAGAAGCCCGGCCAGCCCCCTAAGCTG
CTGATCTATCTGGCTTCCAACCTGGAGAGCGGAGTGCCAGCTAGGTTCTC
TGGCTCCGGCAGCGGCACCGACTTTACCCTGACATACAATCCTGTGGAGG
CCAACGATACAGCTAATTACTATTGCCAGCACTCCGGAGAGCTGCCATAC
ACCTTCGGCGGAGGCACAAAGGTGGAGATCAAGCGTACGGTGGCTGCACC
ATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTA
CAGTGGAAGGTGGATAACGCCCTCCAATCGGGTAACTCCCAGGAGAGTGT
CACAGAGCAGGACAGCAAGGACAGCACCTACAGCCTCAGCAGCACCCTGA
CGCTGAGCAAAGCAGACTACGAGAAACACAAAGTCTACGCCTGCGAAGTC
ACCCATCAGGGCCTGAGCTCGCCCGTCACAAAGAGCTTCAACAGGGGAGA
GTGTTAG

Amino acid sequence of fusion protein Hib-PLT light chain (SEQ ID NO: 73):

| | |
|---|---|
| DIVLTQSPAS LAVSPGQRAT ITCRASKSVH TSGYSYMHWY QQKPGOPPKL LIYLASNLES | 60 |
| GVPARFSGSG SGTDFTLTIN PVEANDTANY YCOHSGELPY TFGGGTKVEI KRTVAAPSVF | 120 |
| IFPPSDEQLK SGTASVVCLL NNFYPREAKV QWKVDNALQS GNSQESVTEQ CSKDSTYSLS | 180 |
| STLTMSKADY EKHKVYACEV THQGLSSPVT KSFNRGEC | 218 |

The nucleic acid sequences and amino acid sequences of the heavy chain and light chain of the fusion protein Hib-PDC are as follows:

Nucleic acid sequence of fusion protein Hib-PDC heavy chain (SEQ ID NO: 74):

CAGGTGCAGCTCGTGGAGAGCGGGGGAGGCGTCGTCCAGCCTGGCCGTAGCCTGCGGCTGGA
CTGTAAGGCTAGCGGAATCACGTTCAGCAACAGCGGAATGCACTGGGTCAGACAGGCTCCTG
GCAAAGGCCTCGAATGGGTCGCCGTGATCTGGTACGACGGGAGCAAGAGATACTACGCAGAT
TCAGTGAAGGGCCGTTTCACAATCAGCCGTGACAACTCGAAGAACACACTGTTCCTGCAGAT
GAACAGCCTGAGGGCAGAAGACACAGCAGTCTACTACTGCGCTACAAATGACGACTACTGGG
GCCAGGGAACACTGGTCACCGTGAGCTCAGCTTCCACCAAGGGCCCATCCGTCTTCCCCCTG
GCGCCCTGCTCCAGGAGCACCTCCGAGAGCACAGCCGCCCTGGGCTGCCTGGTCAAGGACTA
CTTCCCCGAACCGGTGACGGTGTCGTGGAACTCAGGCGCCCTGACCAGCGGCGTGCACACCT
TCCCGGCTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACCAAGGTG
GACAAGAGAGTTGAGTCCGGTGGCGGCGGAAGCGGCGGCGGAGGCAGCGGCGGAGGCGGC
AGC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GGGGGGGGGGGTAGCGGCGGCGGGGGCTCAGGTGGGGGGGGCTCAAAAT
ATGGTCCCCCATGCCCACCATGCCCAGCCCTGAGTTCCTGGGGGGACCATCAGTCTTCCTGT
TCCCCCCAAAACCCAAGGACACTCTCATGATCTCCCGGACCCCTGAGGTCACGTGCGTGGTGG
TGGACGTGAGCCAGGAAGACCCCGAGGTCCAGTTCAACTGGTACGTGGATGGCGTGGAGGTG
CATAATGCCAAGACAAAGCCGCGGGAGGAGCAGTTCAACAGCACGTACCGTGTGGTCAGCGT
CCTCACCGTCCTGCACCAGGACTGGCTGAACGGCAAGGAGTACAAGTGCAAGGTCTCCAACA
AAGGCCTCCCGTCCTCCATCGAGAAAACCATCTCCAAAGCCAAAGGGCAGCCCCGAGAGCCA
CAGGTGTACACCCTGCCCCCATCCCAGGAGGAGATGACCAAGAACCAGGTCAGCCTGACCTG
CCTGGTCAAAGGCTTCTACCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCA
GGCTAACCGTGGACAAGAGCAGGTGGCAGGAGGGGAATGTCTTCTCATGCTCCGTGATGCAT
GAGGCTCTGCACAACCACTACACACAGAAGAGCCTCTCCCTGTCTCFGGGTAAATAG

Note: The underlined and bold sequence is the CD80 nucleic acid sequence.

Amino acid sequence of fusion protein Hib-PDC heavy chain (SEQ ID NO: 68):

| | |
|---|---|
| QVQLVESGGG VVQPGRSLRL DCKASGITFS NSGMHWVRQA PGKGLEWVAV IWYDGSKRYY | 60 |
| ADSVKGRFTI SRDNSKNTLF LQMNSLRAED TAVYYCATND DYWGQGTLVT VSSASTKGPS | 120 |
| VFPLAPCSRS TSESTAALGC LVKDYFPEPV TVSWNSGALT SGVHTFPAVL QSSGLYSLSS | 180 |
| VVTVPSSSLG TKTYTCNVDH KPSNTKVDKR VESGGGGSGG GGSGGGGS [symbol] | 240 |
| [symbol] [symbol] | 300 |
| [symbol] [symbol] | 360 |
| [symbol] [symbol] | 420 |
| [symbol] GGGG SGGGGSGGGG SKYGPPCPPC PAPEFLGGPS VFLEPPKPKD TLMISRTPEV | 430 |
| TCVVVDVSQE DPEVQFNWYV DGVEVHNAKT KPREEQFNST YRVVSVLTVL HQDWLNGKEY | 540 |
| KCKVSNKGLP SSIEKTISKA KGQPREPQVY TLPPSQEEMT KNQVSLTCLV KGFYPSDIAV | 600 |
| EWESNGQPEN NYKTTPPVLD SDGSFFLYSR LTVDKSRWQE GNVFSCSVMH EALHNHYTQK | 660 |
| SLSLSLGK | 668 |

Note: The peptide linker is in italics, and the underlined and hold sequence is the CD80 amino acid sequence.

Nucleic acid sequence of fusion protein Hib-PDC light chain (SEQ ID NO: 75):

GAGATCGTGCTGACACAGAGCCCAGCCACTCTGTCACTGTCCCCAGGAGA
AAGGGCTACTCTGTCTTGCCGGGCAAGCCAGTCTGTCTCCAGCTACCTGG
CCTGGTATCAGCAGAAGCCCGGACAGGCTCCTAGACTGCTGATCTACGAC
GCAAGTAACAGAGCCACCGGCATCCCCGCACGCTTCAGTGGCTCAGGCTC
CGGAACAGACTTTACTCTGACCATCTCTAGTCTGGAGCCTGAAGATTTCG
CCGTGTACTATTGTCAGCAGAGCTCTAATTGGCCTAGAACCTTCGGCCAG
GGCACCAAAGTCGAGATCAAGCGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGTTAG

Amino acid sequence of fusion protein Hib-PDC light chain (SEQ ID NO: 69):

| | |
|---|---|
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ SSNWPRTFGQ GTKVEIKRTV AAPSVFIFPP | 120 |
| SDEQLKSGTA SVVCLLNNFY PREAKVQWKV DNALQSGNSQ ESVTEQDSKD STYSLSSTLT | 130 |
| LSEADYEKHK VYACEVTHQG LSSPVTKSFN RGEC | 214 |

The nucleic acid sequences and amino acid sequences of the heavy chain and light chain of the fusion protein Hib-PDV are as follows:

Nucleic acid sequence of fusion protein Hib-PDV heavy chain (SEQ ID NO: 76):

CAGGTGCAGCTCGTGGAGAGCGGGGGAGGCGTCGTCCAGCCTGGCCGTAGCCTGCGGCTGGA
CTGTAAGGCTAGCGGAATCACGTTCAGCAACAGCGGAATGCACTGGGTCAGACAGGCTCCTG
GCAAAGGCCTCGAATGGGTCGCCGTGATCTGGTACGACGGGAGCAAGAGATACTACGCAGAT
TCAGTGAAGGGCCGTTTCACAATCAGCCGTGACAACTCGAAGAACACACTGTTCCTGCAGAT
GAACAGCCTGAGGGCAGAAGACACAGCAGTCTACTACTGCGCTACAAATGACGACTACTGGG
GCCAGGGAACACTGGTCACCGTGAGCTCAGCTTCCACCAAGGGCCCATCCGTCTTCCCCCTG
GCGCCCTGCTCCAGGAGCACCTCCGAGAGCACAGCCGCCCTGGGCTGCCTGGTCAAGGACTA
CTTCCCCGAACCGGTGACGGTGTCGTGGAACTCAGGCGCCCTGACCAGCGGCGTGCACACCT
TCCCGGCTGTCCTACAGTCCTCAGGACTCTACTCCCTCAGCAGCGTGGTGACCGTGCCCTCCA
GCAGCTTGGGCACGAAGACCTACACCTGCAACGTAGATCACAAGCCCAGCAACACCAAGGTG
GACAAGAGAGTTGAGTCCGGTGGCGGCGGAAGCGGCGGCGGAGGCAGCGGCGGAGGCGGC
AGC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GGCGGGGGGGGTAGCGGCGGCGGGGGCTCAGGTGGGGCTGGGCT
CAAAATATGGTCCCCCATGCCCACCATGCCCAGCACCTGAGTTCCTGGGGGGACCATCAGTCT
TCCTGTTCCCCCCAAAACCCAAGGACACTCTCATGATCTCCCGGACCCCTGAGGTCACGTGCG
TGGTGGTGGACGTGAGCCAGGAAGACCCCGAGGTCCAGTTCAACTGGTACGTGGATGGCGTG
GAGGTGCATAATGCCAAGACAAAGCCGCGGGAGGAGCAGTTCAACAGCACGTACCGTGTGGT
CAGCGTCCTCACCGTCCTGCACCAGGACTGGCTGAACGGCAAGGAGTACAAGTGCAAGGTCT
CCAACAAAGGCCTCCCGTCCTCCATCGAGAAAACCATCTCCAAAGCCAAAGGGCAGCCCCGA
GAGCCACAGGTGTACACCCTGCCCCCATCCCAGGAGGAGATGACCAAGAACCAGGTCAGCCT
GACCTGCCTGGTCAAAGGCTTCTACCCCAGCGACATCGCCGTGGAGTGGGAGAGCAATGGGC
AGCCGGAGAACAACTACAAGACCACGCCTCCCGTGCTGGACTCCGACGGCTCCTTCTTCCTCT
ACAGCAGGCTAACCGTGGACAAGAGCAGGTGGCAGGAGGGGAATGTCTTCTCATGCTCCGTG
ATGCATGAGGCTCTGCACAACCACTACACACAGAAGAGCCTCTCCCTGTCTCTGGGTAAATAG

Note: The underlined and bold sequence is the VEGFR nucleic acid sequence.

Amino acid sequence of fusion protein Hib-PDV heavy chain (SEQ ID NO: 70):

| | |
|---|---|
| QVQLVESGGG VVQPGRSLRL DCKASGITFS NSGMHWVRQA PGKGLEWVAV IWYDGSKRYY | 60 |
| ADSVKGRFTI SRDNSENTLF LQMNSLRAED TAVYYCATND DYWGQGTLVT VSSASTKGPS | 120 |
| VFPLAPCSRS TSESTAALGC LVKDYFPEPV TVSWNSGALT SGVHTFPAVL QSSGLYSLSS | 180 |
| VVTVPSSSLG TKTYTCNVDH KPSNTKVDKR VESGGGGSGG GGSGGGGS [symbol] | 240 |
| [symbol] [symbol] | 300 |
| [symbol] [symbol] | 360 |
| [symbol] [symbol] | 420 |
| [symbol] GGGGSGGGGS GGGGSKYGPP CPPCPAPEFL GGPSVFLFPP KPKDTLMISR | 480 |
| TPEVTCVVVD VSQEDPEVQF NWYVDGVEVH NAKTEPREEQ FNSTYRVVSV LTVLHQDWLN | 540 |
| GKEYKCKVSN KGLPSSIEKT ISKAKGOPRE PQVYTLPPSQ EEMTKNQVSL TCLVKGFYPS | 600 |
| DIAVEWESNG QPENNYKTTP PVLDSDGSFF LYSRLTVDKS RWQEGNVESC SVMHEALHNH | 660 |
| YTQKSLSLSL GK | 672 |

Note: The peptide linker is in italics, and the underlined and bold sequence is the VEGF amino acid sequence.

Nucleic acid sequence of fusion protein Hib-PDV light chain (SEQ ID NO: 77):

GAGATCGTGCTGACACAGAGCCCAGCCACTCTGTCACTGTCCCCAGGAGA
AAGGGCTACTCTGTCTTGCCGGGCAAGCCAGTCTGTCTCCAGCTACCTGG
CCTGGTATCAGCAGAAGCCCGGACAGGCTCCTAGACTGCTGATCTACGAC
GCAAGTAACAGAGCCACCGGCATCCCCGCACGCTTCAGTGGCTCAGGCTC
CGCTAACAGACTTTACTCTGACCATCTCTAGTCTGGAGCCTGAAGATTTC
GCCGTGTACTATTGTCAGCAGAGCTCTAATTGGCCTAGAACCTTCGGCCA
GGGCACCAAAGTCGAGATCAAGCGTACGGTGGCTGCACCATCTGTCTTCA
TCTTCCCGCCATCTGATGAGCAGTTGAAATCTGGAACTGCCTCTGTTGTG
TGCCTGCTGAATAACTTCTATCCCAGAGAGGCCAAAGTACAGTGGAAGGT
GGATAACGCCCTCCAATCGGGTAACTCCCAGGAGAGTGTCACAGACTCAG
GACAGCAAGGACAGCACCTACAGCCTCAGCAGCACCCTGACGCTGAGCAA
AGCAGACTACGAGAAACACAAAGTCTACGCCTGCGAAGTCACCCATCAGG
CCCTGAGCTCGCCCGTCACAAAGAGCTTCAACAGGGGAGAGTGTTAG

Amino acid sequence of fusion protein Hib-PDV light chain (SEQ ID NO: 71):

| | |
|---|---|
| EIVLIQSPAT LSLSPGERAT LSCRASQSVS SYLAWYQQKP GQAPRLLIYD ASNRATGIPA | 60 |
| RFSGSGSGTD FTLTISSLEP EDFAVYYCQQ SSNWPRTFGQ GTKVEIKRTV AAPSVFIFPP | 120 |
| SDEQLKSGTA SVVCLLNNFY PREAKVQWKV DNALQSGNSQ ESVTEQDSKD STYSLSSTLT | 180 |
| LSKADYEKHK VYACEVTHQG LSSPVTKSFN RGEC | 214 |

The amino acid sequences of the heavy chain and light chain of the Control protein 1

Amino acid sequence of Control protein 1 heavy chain (SEQ ID NO: 124):

| | |
|---|---|
| QVQLQESGPG LVKPSETLSL TCTVSGFSLS RYSVHWIRQP PGKGLEWLGM IWGVGTTDYN | 60 |
| SALKSRLTIS KDTSKNQFSL KLSSVTAADT AVYYCARNWG TADYFDYWGQ GTTVTVSSAS | 120 |
| TKGPSVFPLA PSSKSTSGGT AALGCLVKDY FPEPVTVSWN SGALTSGVHT FPAVLQSSGL | 180 |
| YSLSSVVTVP SSSLGTQTYI CNVNHKPSNT KVDKKVEPKS CDKTHTCPPC PAPELLGGPS | 240 |
| VFLEPPKPKD TLMISRTPEV TCVVVDVSHE DPEVKFNWYV DGVEVHNAKT KPREEQYNST | 300 |
| YRVVSVLTVL HQDWLNGKEY KCKVSNKALP APIEKTISKA KGQPREPQVY TLPPSREEMT | 360 |
| KNQVSLTCLV KGFYPSDIAV EWESNGQPEN NYKTTPPVLD SDGSFFLYSK LTVDKERWQQ | 420 |
| GNVFSCSVMH EALHNHYTQK SLSLSPGKGG GGSGGGGSGG GGS [symbol] | 480 |
| [symbol] [symbol] | 540 |
| [symbol] [symbol] | 600 |

Note: The peptide linker is in italics, and the underlined and bold sequence is the TGF-βRII amino acid sequence.

Amino acid sequence of Control protein 1 light chain (SEQ ID NO: 125):

| | |
|---|---|
| DIVLTQSPAS LAVSPGQRAT ITCRASKSVH TSGYSYMHWY QQKPGQPPKL LIYLASNLES | 60 |
| GVPARFSGSG SGTDFTLTIN PVEANDTANY YCQHSGELPY TFGGGTKVEI KRTVAAPSVF | 120 |
| IFPPSDEQLK SGTASVVCLL NNFYPREAKV QWNVDNALQS GNSQESVTEQ DSKDSTYSLS | 180 |
| STLTLSKADY EKHKVYACEV THQGLSSPVT KSFNRGEC | 218 |

The amino acid sequences of the heavy chain and light chain of the Control protein 2

Amino acid sequence of Control protein 2 heavy chain (SEQ ID NO: 126):

| | |
|---|---|
| QVQLVESGGG VVQPGRSLRL DCKASGITFS NSGMHWVRQA PGKGLEWVAV IWYDGSERYY | 60 |
| ADSVKGRFTI SRDNSKNTLF LQMNSLRAED TAVYYCATND DYWGQGTLVT VSSASTKGPS | 120 |
| VFPLAPCSRS TSESTAALGC LVKDYFPEPV TVSWNSGALT SGVHTFPAVL QSSGLYSLSS | 130 |
| VVTVPSSSLG TKTYTCNVDH KPSNTKVDKR VESKYGPPCP PCPAPEFLGG PSVELEPPKP | 240 |
| KDTLMTSRTP EVTCVVVDVS QEDPEVQFNW YVDGVEVHNA KTKPREEQFN STYRVVSVLT | 300 |
| VLHQDWLNGK EYKCKVSNKG LPSSIEKTIS KANGQPREPQ VYTLPPSQEE MTKNQVSLTC | 360 |
| LVKGEYPSDI AVEWESNGQP ENNYKTTPPV LDSDGSFFLY SRLTVDKSRW QEGNVESCSV | 420 |
| MHEALHNHYT QKSLSLSLGK GGGGSGGGGS GGGGS [symbol] | 430 |
| [symbol] [symbol] | 540 |
| [symbol] [symbol] | 600 |
| [symbol] [symbol] | 653 |

Note: The peptide linker is in italics, and the underlined and bold sequence is the CD80 amino acid sequence.

Amino acid sequence of Control protein 2 light chain (SEQ ID NO: 127):

| | |
|---|---|
| EIVLTQSPAT LSLSPGERAT LSCRASQSVS SYLAWYQQKP GQAPRLLIYD ASNRATGIPA | 60 |
| RFSGSGSGTD FILTISSLEP EDFAVYYCQQ SSNWPRTFGQ GTKVEIKRTV AAPSVFIFPP | 120 |
| SDEQLKSGTA SVVCLLNNEY PREAKVQWKV DNALQSGNSQ ESVTEQDSKD STYSLSSTLT | 130 |
| LSKADYEKHK VYACEVTHQG LSSPVTKSFN RGEC | 214 |

The amino acid sequences of the heavy chain and light chain of the Control protein 3

Amino acid sequence of Control protein 3 heavy chain (SEQ ID NO: 128):

| | |
|---|---|
| QVQLVESGGG VVQPGRSLRL DCKASGITFS NSGMHWVRQA PGKGLEWVAV IWYDGSKRYY | 60 |
| ADSVKGRFTI SRDNSKNTLF LQMNSLRAED TAVYYCATND DYWGQGTLVT VSSASTKGPS | 120 |
| VFPLAPCSRS TSESTAALGC LVKDYFPEPV TVSWNSGALT SGVHTEPAVL QSSGLYSLSS | 180 |
| VVTVPSSSLG TKTYTCNVDH KPSNTKVDKR VESKYGPPCP PCPAPEFLGG PSVFLFPPKP | 240 |
| KDTLMISRTP EVTCVVVDVS QEDPEVQFNW YVDGVEVHNA KTKPREEQFN STYRVVSVLT | 300 |
| VLHQDWLNGK EYKCHVSNKG LPSSIEKTIS KAKGQPREPQ VYTLPPSQEE MTKNQVSLTC | 260 |
| LVKGFYPSDI AVEWESNGQP ENNYKTTPPV LDSDGSFFLY SRLTVDKSRW QEGNVFSCSV | 420 |
| MHEALHNHYT QKSLSLSLGK GGGGSGGGGS GGGGSGRPFV EMYSEIPEII HMTEGRELVI | 480 |
| PCRVTSPNIT VTLKKFPLDT LIPDGKRIIW DSRKGFIISN ATYKEIGLLT CEATVNGHLY | 540 |
| KTNYLTHRQT NTIIDVVLSP SHGIELSVGE KLVLNCTART ELNVGIDFNW EYPSSKHQHK | 600 |
| KLVNRDLKTQ SGSEMKKFLS TLTIDGVTRS DQGLYTCAAS SGLMTKKNST FVRVHEP | 657 |

Note: The peptide linker is in italics, and the underlined and bold sequence is the VEGFR amino acid sequence.

Amino acid sequence of Control protein 3 light chain (SEQ ID NO: 129):

| | |
|---|---|
| EIVLTQSPAT LSLSPGERAT LSCRASQSVS SYLAWYQQKP GQAPRLLIYD ASNRATGIPA | 60 |
| RFSGSGSGTD FTLTISSLEP EDFAVYYCQQ SSNWPRTFGQ GTKVEIKRTV AAPSVFIFPP | 120 |
| SDEQLKSGTA SVVCLLNNFY PREAKVQWKV DNALQSGNSQ ESVTEQDSKD STYSLSSTLT | 180 |
| LSKADYEKHK VYACEVTHQG LSSPVTKSFN RGEC | 214 |

TGF-β-R-His

TGF-β-R-His is the His-tagged TGF-βRII extracellular region fragment, wherein the amino acid sequence of the TGF-βRII extracellular region fragment (SEQ ID NO: 137) is as follows:

| | |
|---|---|
| TIPPHVQKSV NNDMIVTDNN GAVKFPQLCK FCDVRFSTCD NQKSCMSNCS ITSICEKPQE | 60 |
| VCVAVWRKND ENITLETVCH DPKLPYHDFI LEDAASPKCI MKEKKKPGET FFMCSCSSDE | 120 |
| CNDNIIFSEE YNTSNPD | 137 |

VEGF-R-His Amino Acid Sequence

VEGF-R-His is the His-tagged combination fragment of the second extracellular region of VEGFR1 and the third extracellular region of VEGFR2, wherein the amino acid sequence of combination fragment of the second extracellular region of VEGFR1 and the third extracellular region of VEGFR2 (SEQ ID NO: 130) is as follows:

| | |
|---|---|
| GRPFVEMYSE IPEIIHMTEG RELVIPCRVT SPNITVILKK FPLDTLIPDG KRIIWDSRKG | 60 |
| FIISNATYKE IGLLTCEATV NGHLYKTNYL THRQTNTIID VVLSPSHGIE LSVGEKLVLN | 120 |
| CTARTELNVG IDFNWEYPSS KHQHKKLVNR DLKTQSGSEM KKFLSTLTID GVTRSDQGLY | 180 |
| TCAASSGLMT KKNSTFVRVH EP | 202 |

CD80-his Amino Acid Sequence

CD80-His is the His-tagged CD80 fragment, wherein the amino acid sequence of the CD80 fragment (SEQ ID NO: 32) is as follows:

| | |
|---|---|
| VIHVTKEVKE VATLSCGENV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADFPT PSISDFEIPT | 120 |
| SNIRRIICST SGGFPEPHLS WLENGEELNA INTTVSQDPE TELYAVSSKL DFNMTTNHSF | 180 |
| MCLIKYGHLR VNQTFNWN | 198 |

### Example 2 CHO Expression

A. Cell Thawing and Expansion:

CHO host cells were thawed with a culture volume of 10 mL and culture conditions of 370° C., 200 rpm, 8% CO₂, and 80% humidity. After thawing, the cell density was 2.82×10⁵ cells/mL, and the viability was 95.41%.

After 72 hours of cell expansion and culture, the density of viable cells was 2.22×10⁶ cells/mL, and the viability was 98.32%.

The cells were re-seeded at 5×1.0⁵ cells/ml, for cell re-seeding operation. After 48 hours of continuous culture, the density of viable cells was 2×10⁶ cells/mL, and the viability was 98.68%.

The cells were seeded into a 6-well plate at 5×10⁵ cells/well, with a culture volume of 2 mL/well, and placed in a carbon dioxide incubator (culture conditions: 37° C., 8% CO₂) overnight for adherence and transfection the next day.

B. Transfection

The transfection reagent used Lipofectamine® LTX Reagent (Invitrogen, catalog No. 1.5338-100), containing LTX and Plus reagents.

Transfection procedure: Changing the medium in the 6-well plate and preparing the transfection complex: LTX: 9 μl/well+plasmid: 2.5 μg/well+plus: 2.5 μl/well. The transfection complex was added at 250 μl/well to the 6-well plate. The medium in each well of the 6-well plate was changed 5 hours after cell transfection. After 48 hours of transfection, the supernatant was taken to detect the antibody content of the supernatant by the double antigen ELISA method.

C. Batch Culture and Fed-Batch Culture

After 48 hours of transfection, the cells in the six-well plate were digested, collected into a cell culture tube, and adapted to grow in suspension at a cell density of 3×10⁵ cells/mL. When the cell viability returned to about 90%, Hib-PLT expressing cell pool and the Control protein 1 expressing cell pool were cultured in batch culture, and Hib-PDC, Control protein 2, Hib-PDV, and Control protein 3 were cultured in fed-batch culture.

Batch culture: Cells were cultured in a TPP cell culture tube at a cell density of 3×10⁵ cells/mL, with a volume of 30 mL and culture conditions at 37.0° C., 200 rpm, 8% CO₂, and 80% humidity, and the cell viability and metabolism were monitored every day. The culture was terminated until the viability was <60%, and the cell supernatant after the completion of the culture was taken for concentration detection.

Fed-batch culture: Cells were cultured in a TPP cell culture tube at a cell density of 3×10⁵ cells/mL, with a volume of 30 mL and culture conditions at 37.0° C., 200 rpm, 8% CO₂, and 80% humidity. The cell concentrated medium was given according to a certain fed-batch strategy (see Table 5), during which Glc was controlled at 2-6 g/L. The culture was terminated until the viability was <60%, and the cell supernatant after the completion of the culture was taken for concentration detection.

TABLE 5
Fed-batch strategy

| | |
|---|---|
| Day 0 | 3 × 10⁵/ml, culture volume 30 ml |
| Day 4, 6, 8, 10, 12 | 900 μL CellBoost7A, 90 μL CellBoost7B |
| Day 16 | Detecting VCD and viability, stocking the |
| | sample and terminating the experiment |

Table 6 shows the expression level results of Hibody bispecific fusion proteins Hib-PLT, Hib-PDC and Hib-PDV and their corresponding control proteins. The results show that the relative value of the increase in Hib-PLT expression level increased by 52.85% compared with its corresponding control protein; the relative value of the increase in Hib-PDC expression level increased by 33.43% compared with its corresponding control protein; and the relative value of the increase in Hib-PDV expression level increased by 25.6% compared with its corresponding control protein, indicating the expression level of the Hibody bispecific fusion protein provided in the present disclosure is significantly superior to that of the corresponding control protein, and the bispecific antibody provided in the present disclosure has unexpected technical effects.

TABLE 6
Expression level of bispecific antibodies

| | | | Relative |
|---|---|---|---|
| Category | Method | Expression level | value of increase |
| Hib-PLT | Batch culture | 188 mg/L | 52.85% |
| Control protein 1 | Batch culture | 123 mg/L | |
| Hib-PDC | Fed-batch culture | 349.59 mg/L | 33.43% |
| Control protein 2 | Fed-batch culture | 262 mg/L | |
| Hib-PDV | Fed-batch culture | 243.95 mg/L | 25.6% |
| Control protein 3 | Fed-batch culture | 194.72 mg/L | |

Note:
Relative value of increase = (the expression level of the bispecific fusion protein provided in the present disclosure − the expression level of the corresponding control protein)/the expression level of the corresponding control protein.

### Example 3 Determination of Binding Activity

1. Determination of Hib-PLT Binding Activity:

(1) Coating antigen: TGF-β1 was diluted to 2 μg/ml with coating buffer, added to a 96-well plate at 100 and allowed to stand overnight at 2-8° C.;

(2) Washing the plate: The plate was automatically washed 3 times at 350 μl/well with PBST used as the eluent, and was patted to dryness after washed;

(3) Blocking the plate: The plate was blocked at 300 μl/well with 3% BSA-PBST used as the blocking solution, and incubated at 37° C. for 2 hours;

(4) Washing the plate: The plate was automatically washed 3 times at 350 with PBST used as the eluent, and was patted to dryness after washed;

(5) Adding samples: Each concentration point of samples is as follows. The sample with each concentration point was sequentially added to a 96-well plate at 100 μl/well, and each concentration point was in parallel with 2 replicate wells, and the plate was incubated at 37° C. for 2 hours;

| Test group | S01 | S02 | S03 | S04 | S05 | S06 | S07 | S08 | S09 | S10 | S11 |
|---|---|---|---|---|---|---|---|---|---|---|---|
| Hib-PLT | 555.556 | 55.556 | 5.556 | 1.852 | 0.617 | 0.206 | 0.069 | 0.023 | 0.008 | 0.001 | 0.000 |
| TGF-β-R-His | 64516.000 | 6451.600 | 645.160 | 215.053 | 71.684 | 23.895 | 7.965 | 2.655 | 0.885 | 0.088 | 0.009 |

(Unit: nM)

(6) Washing the plate: The plate was automatically washed 3 times at 350 μl/well with PBST used as the eluent, and was patted to dryness after washed;

(7) Adding the secondary antibody:

Hib-PLT sample well: Goat-anti-Human-Fc-HRP was diluted at 1:5000, and added at 100 μl/well. Then the plate was incubated at 37° C. for 2 hours;

TGF-β-R-His well: Anti-His-HRP was diluted at 1:5000, and added at 100 μl/well. Then the plate was incubated at 37° C. for 2 hours;

(8) Washing the plate: The plate was automatically washed 5 times at 350 μl/well with PBST used as the eluent, and was patted to dryness after washed;

(9) Color development: The plate was subjected to color development for 2 minutes at 100 μl/well with TMB used as the color development solution;

(10) Terminating the color development: The plate was terminated for color development at 50 μl/well with 2M H₂SO₄ used as the stop solution;

(11) Reading: The absorbance values were read at 450/655 nm respectively using the MD microplate reader. The concentration of the sample to be tested was used as the abscissa and the absorbance value as the ordinate to plot making a 4-PL curve, and the software automatically gave the EC50 value.

2. Determination of Hib-PDV Binding Activity:

(1) Coating antigen: VEGF was diluted to 1 μg/ml with coating buffer; added to a 96-well plate at 100 μl/well, and allowed to stand overnight at 2-8° C.;

(2) Washing the plate: The plate was automatically washed 3 times at 350 μl/well with PBST used as the eluent, and was patted to dryness after washed;

(3) Blocking the plate: The plate was blocked at 300 μl/well with 3% BSA-PBST used as the blocking solution, and incubated at 37° C. for 2 hours;

(4) Washing the plate: The plate was automatically washed 3 times at 350 μl/well with PBST used as the eluent, and was patted to dryness after washed;

(5) Adding samples: Each concentration point of samples is as follows. The sample with each concentration point was sequentially added to a 96-well plate at 100 μl/well, and each concentration point was in parallel with 2 replicate wells, and the plate was incubated at 37° C. for 2 hours;

| Test group | S01 | S02 | S03 | S04 | S05 | S06 | S07 | S08 | S09 | S10 | S11 |
|---|---|---|---|---|---|---|---|---|---|---|---|
| Hib-PDV | 555.556 | 55.556 | 5.556 | 1.852 | 0.617 | 0.206 | 0.069 | 0.023 | 0.005 | 0.001 | 0.000 |
| VEGFR-His | 43478.300 | 4347.830 | 434.783 | 144.928 | 48.309 | 16.103 | 5.368 | 1.789 | 0.358 | 0.036 | 0.004 |

(Unit: nM)

(6) Washing the plate: The plate was automatically washed 3 times at 350 μl/well with PBST used as the eluent, and was patted to dryness after washed;

(7) Adding the secondary antibody:

Hib-PDV sample well: Goat-anti-Human-Fc-HRP was diluted at 1:5000, and added at 100 μl/well. Then the plate was incubated at 37° C. for 2 hours;

VEGFR-His well: Anti-His-HRP was diluted at 1:5000, and added at 100 μl/well. Then the plate was incubated at 37° C. for 2 hours;

(8) Washing the plate: The plate was automatically washed 5 times at 350 μl/well with PBST used as the eluent, and was patted to dryness after washed;

(9) Color development: The plate was subjected to color development for 2 minutes at 100 μl/well with TMB used as the color development solution;

(10) Terminating the color development: The plate was terminated for color development at 50 μl/well with 2M H₂SO₄ used as the stop solution;

(11) Reading: The absorbance values were read at 450/655 nm respectively using the MD microplate reader. The concentration of the sample to be tested was used as the abscissa and the absorbance value as the ordinate to plot making a 4-PL curve, and the software automatically gave the EC50 value.

3. Determination of Hib-PDC Binding Activity:

(1) Coating antigen: CTLA-4 Protein was redissolved with 1 mL of sterilized water at a concentration of 200 μg/mL, diluted to 20 μg/ml with coating buffer, added to a 96-well plate at 100 and allowed to stand overnight at 2-8° C.;

(2) Washing the plate: The plate was automatically washed 3 times at 350 μl/well with PBST used as the eluent, and was patted to dryness after washed;

(3) Blocking the plate: The plate was blocked at 300 μl/well with 3% BSA-PBST used as the blocking solution, and incubated at 37° C. for 2 h±20 min;

(4) Washing the plate: The plate was automatically washed 3 times at 350 μl/well with PBST used as the eluent, and was patted to dryness after washed;

(5) Adding samples: The biotinylated sample was diluted with PBST. Each concentration point of samples was as follows. The sample with each concentration point was sequentially added to a 96-well plate at 100 μl/well, and each concentration point was in parallel with 2 replicate wells, with the blank well being PBST, and the plate was incubated at 37° C. for 2 h±20 min;

| Test group | S01 | S02 | S03 | S04 | S05 | S06 | S07 | S08 | S09 | S10 | S11 |
|---|---|---|---|---|---|---|---|---|---|---|---|
| Hib-PDC | 1000000 | 100000 | 10000 | 3333.333 | 1111.111 | 370.370 | 123.457 | 41.152 | 13.717 | 1.372 | 0.137 |
| CD80-His | 1000000 | 100000 | 10000 | 3333.333 | 1111.111 | 370.370 | 123.457 | 41.152 | 13.717 | 1.372 | 0.137 |

(Unit: nM)

(6) Washing the plate: The plate was automatically washed 3 times at 350 with PBST used as the eluent, and was patted to dryness after washed;

(7) Adding the secondary antibody:

Hib-PDC sample well and CD80-His well: Streptavidin-HRP was diluted at 1:2000, and added at 100 μl/well. Then the plate was incubated at 37° C. for 1 h±10 min;

(8) Washing the plate: The plate was automatically washed 5 times at 350 μl/well with PBST used as the eluent, and was patted to dryness after washed;

(9) Color development: TMB Double Slow substrate was added at 100 μl/well and the plate was subjected to color development for 6 minutes at room temperature shielded from light;

(10) Terminating the color development: The plate was terminated for color development at 50 μl/well with 2M H₂SO₄ used as the stop solution;

(11) Reading: The absorbance values were read at 450/655 nm respectively using the MD microplate reader. The concentration of the sample to be tested was used as the abscissa and the absorbance value as the ordinate to plot making a 4-PL curve, and the software automatically gave the EC50 value. (see table 7).

TABLE 7
EC50 value of each test group

| Test group | EC50 value |
|---|---|
| Hib-PLT | 0.164 nM |
| TGF-β-R-His | 11.60 nM |
| Hib-PDV | 0.061 nM |
| VEGFR-His | 2154 nM |
| Hib-PDC | 24.36 nM |
| CD80-His | 50.69 nM |

The results of determination of binding activity showed that, comparison between Hib-PLT and TGF-β receptor monomer: Hib-PLT has an EC50 value of 0.164 nM, TGF-β receptor monomer has an EC50 value of 11.60 nM, and the binding activity of Hib-PLT is about 70 times better than that of TGF-β receptor monomer; comparison between Hib-PDV and VEGF receptor monomer: Hib-PDV has an EC50 value of 0.061 nM, VEGF receptor monomer has an EC50 value of 2154 nM, and the binding activity of Hib-PDV binding activity is about 35,311 times better than that of VEGF receptor monomer; comparison between Hib-PDC and CD80 monomer: Hib-PDC has an EC50 value of 24.36 nM, CD80 monomer has an EC50 value of 50.69 nM, and the binding activity of Hib-PDC is about 2 times better than that of CD80 monomer. That is, the binding activities of Hib-PLT, Hib-PDV, and Hib-PDC constructed with the bi-antibody model provided by the present disclosure are much better than that of their corresponding monomers.

### Example 4 the Efficacy of Hib-PDV on Subcutaneously Transplanted Tumor of Colon Cancer MC38 Cells in C57BL/6J-hPD-1 Mice

Thirty-two 7-week-old, C57BL/6.1 humanized PD-1 transgenic mice (from Shanghai Model Organisms Center, Inc.) were collected, 0.1 mL of colon cancer MC38 cell suspension (1×10⁶ cells/mouse) was inoculated subcutaneously into the right flank of mice. When the tumor grew to about 100 mm³, mice were randomly divided into groups according to the tumor size, namely Vehicle (PBS) group, Hib-PDV (1.25 mg/kg) group, Hib-PDV (2.5 mg/kg) group and Hib-PDV (5 mg/kg) group, respectively, a total of 4 groups, 8 tumor-bearing mice in each group. They were administered intraperitoneally once a week, a total of 3 administrations, during the administration period, the long diameter and short diameter of the tumor and body weight were measured twice a week. On the 21^{st} day after administration, the tumor growth inhibition rate (TGI_{RTV}) was calculated for drug efficacy evaluation, as shown in Table 8.

TABLE 8
Dosage regimen of Hib-PDV on
tumor inhibitory activity of mouse colon cancer

| | Admin- | | Admin- | Admin- | Admin- |
|---|---|---|---|---|---|
| | istration | Dosage | istration | istration | istration |
| Group | group | (mg/kg) | route | frequency | volume |
| 1 | Vehicle | — | i.p. | QW × 3 | 10 mL/kg |
| 2 | Hib-PDV | 5 | i.p. | QW × 3 | 10 mL/kg |
| 3 | Hib-PDV | 2.5 | i.p. | QW × 3 | 10 mL/kg |
| 4 | Hib-PDV | 1.25 | i.p. | QW × 3 | 10 mL/kg |

Calculation Formula:

Tumor volume: TV=D₁×D₂²/2, where D1 and D2 represent the long diameter and short diameter of the tumor, respectively;

Relative tumor volume: RTV=T_{VT}/T_{V1}, where T_{V1} is the tumor volume before administration, and T_{VT} is the tumor volume at each measurement;

Relative tumor inhibition rate: TGI_{RTV}(%)=(1−T_{RTV}/C_{RTV})×100%, where T_{RTV} represents the RTV of the administration group or the positive control group, and C_{RTV} represents the RTV of the negative control group.

Results and conclusions: The tumor inhibitory effects of Hib-PDV on mouse colon cancer model are shown in Table 9 and FIG. 2, where Table 9 shows the tumor volume and tumor growth inhibition rate of Hib-PDV on mouse colon cancer model, and FIG. 2 is a graph of tumor growth after administration. Experimental results show that Hib-PDV has a significant inhibitory effect on mouse colon cancer model tumor.

TABLE 9
effect of Hib-PDV on the tumor volume and tumor
inhibition rate of mouse colon cancer model (mean + SEM)

| | Tumor volume (TV, mm³) | | |
|---|---|---|---|
| Administration group | D0 | D21 | TGIRTV (%) |
| Vehicle | 86 + 10 | 1299 + 174 | — |
| Hib-PDV (5 mg/kg) | 86 + 10 | 675 + 218 | 46 |
| Hib-PDV (2.5 mg/kg) | 86 + 8 | 755 + 305 | 47 |
| Hib-PDV (1.25 mg/kg) | 85 + 8 | 1088 + 259 | 14 |

### Example 5 the Efficacy of Hib-PDC on Subcutaneously Transplanted Tumor of Transgenic hPD-L1 MC38 Cells in C57BL/6J-hPD-1 Mice

Thirty-two 7-9 week-old, humanized C57BL/6J humanized PD-1 transgenic female mice (from Shanghai Model Organisms Center, Inc.) were collected. 0.1 mL of transgenic hPD-L1 MC38 cell suspension (1×10⁶ cells/mouse) was subcutaneously implanted in the back of the right forelimb of the mouse. When the tumor grew to about 100 mm³, mice were randomly divided into groups according to the tumor size, namely Vehicle (PBS) group, Hib-PDC (1.25 mg/kg) group, Hib-PDC (2.5 mg/kg) group and Hib-PDC (5 mg/kg) group, respectively, a total of 4 groups, 8 tumor-hearing mice in each group. They were administered intraperitoneally once a week, a total of 3 administrations, during the administration period, the long diameter and short diameter of the tumor and body weight were measured twice a week. On the 21st day after administration, the tumor growth inhibition rate (TGI_{RTV}) was calculated for drug efficacy evaluation, as shown in Table 10,

TABLE 10
Dosage regimen of Hib-PDC on tumor
inhibitory activity of mouse colon cancer

| | Admin- | | Admin- | Admin- | Admin- |
|---|---|---|---|---|---|
| | istration | Dosage | istration | istration | istration |
| Group | group | (mg/kg) | route | volume | frequency |
| 1 | Vehicle(PBS) | — | i.p. | 10 ml/kg | QW × 3 |
| 2 | Hib-PDC | 1.25 | i.p. | 10 ml/kg | QW × 3 |
| 3 | Hib-PDC | 2.5 | i.p. | 10 ml/kg | QW × 3 |
| 4 | Hib-PDC | 5 | i.p. | 10 ml/kg | QW × 3 |

Calculation Formula:

Tumor volume: TV=D₁×D₂²/2, where D1 and D2 represent the long diameter and short diameter of the tumor, respectively;

Relative tumor volume: RTV=T_{VT}/T_{V1}, where T_{V1} is the tumor volume before administration, and T_{VT} is the tumor volume at each measurement;

Relative tumor inhibition rate: TGI_{RTV}(%)=(1−T_{RTV}/C_{RTV})×100%, where T_{RTV} represents the RTV of the administration group or the positive control group, and C_{RTV} represents the RTV of the negative control group.

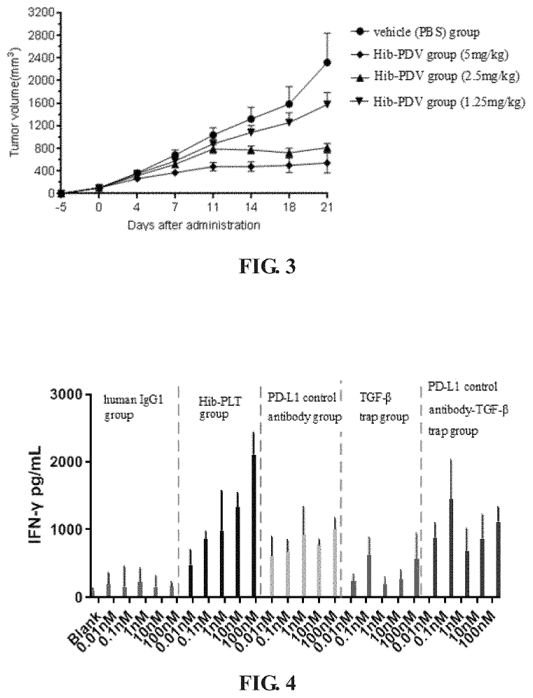

Results and conclusions: The tumor inhibitory effects of Hib-PDC on mouse colon cancer are shown in Table 11 and FIG. 3, where Table 11 shows the effect of Hib-PDC on the tumor volume and tumor inhibition rate on mouse colon cancer model, and FIG. 3 is a graph of tumor growth after administration. Experimental results show that Hib-PDC has a significant inhibitory effect on subcutaneously transplanted tumors of colorectal cancer in mice.

TABLE 11
effect of Hib-PDC on the tumor volume and tumor
inhibition rate of mouse colon cancer model (mean + SEM)

| | Dosage | Tumor volume (TV, mm³) | | |
|---|---|---|---|---|
| Group | (mg/kg) | D0 | D21 | TGIRTV (%) |
| PBS | — | 101 ± 10 | 2319 ± 517 | — |
| Hib-PDC | 1.25 | 101 ± 8 | 1577 ± 211 | 31 |
| Hib-PDC | 2.5 | 101 ± 9 | 813 ± 74 | 63 |
| Hib-PDC | 5 | 101 ± 9 | 541 ± 179 | 77 |

### Example 6 Effect of Hib-PLT on the Cytokine Secretion in the Mixed Reaction of Allogeneic Lymphocytes

ELBA method was used to detect the concentration of cytokine IFN-γ in the cell supernatant to evaluate the role of Hib-PLT in activating CD4⁺ cells. The mononuclear lymphocytes from donor 1 were induced into mature dendritic cells in vitro by DC cell rapid maturation in vitro kit (cat: CT-004, StemEry), the cells were collected after 48 h and diluted to 2×10⁵ cells/mL. The CD4⁺ T cells from donor 2 were enriched with Magnetic beads (EasySep™ Human CD4⁺ T Cell Enrichment Kit, StemCell, cat: 19052) and diluted to 1×1.0⁶ cells/mL. The mature dendritic cells and CD4⁺ T cells were mixed at a volume ratio of 1:1, and the mixed cell suspension was added to a 96-well plate at a volume of 100 μL/well. Hib-PLT, PD-L1 control antibody+TGF-β Trap (combination medication), PD-L1 control antibody, TGF-β Trap, and human IgG1 (Biolegend) were added to a 96-well plate containing mixed cell suspension at a final concentration of 0.01-100 nM. The plate was then placed in a 37° C., 5% CO₂ incubator for 120 h, centrifuged at 2000 rpm for 5 min, and 150-200 μL of the cell supernatant was collected. ELISA kits (Human IL-2 Quantikine ELISA Kit, R&D, cat: 82050; Human IFN-gamma Quantikine ELISA Kit, R&D, cat: SIF50C) were used to detect IFN-γ factor in the supernatant of the mixed reaction of allogeneic lymphocytes. One-way ANOVA was used to analysis the differences between groups.

Results and conclusions: As shown in FIG. 4, the level of Hib-PLT at a concentration of 10-100 nM promoting cell secretion of IFN-γ factor was significantly higher than that of the combination medication group of PD-L1 control antibody and TGF-β trap (p<0.05). The results show that at the concentration level of 10-100 nM. Hib-PLT is better than the combination medication group in promoting the activation of CD4⁺ T cells in the mixed lymphocytes reaction.

The present disclosure has been exemplified by various specific examples. However, a person of ordinary skill in the art can understand that the present disclosure is not limited to each specific embodiments, and a person of ordinary skill can make various changes or modifications within the scope of the present disclosure, and each technical feature mentioned in various places in this specification can be combined with each other without departing from the spirit and scope of the present disclosure. Such changes and modifications are within the scope of the present disclosure.

1. A bispecific fusion protein, wherein the fusion protein has a structure in which a second binding domain is inserted in the hinge region of a full-length immunoglobulin G (IgG) through an optional peptide linker, and the Fab region of the IgG is a first binding domain; after inserting the second binding domain, the heavy chain of the IgG is the heavy chain of the fusion protein, and the light chain of the IgG is the light chain of the fusion protein; the second binding domain is selected from a human receptor or ligand, a fragment of the receptor or ligand, and a fragment combination of the receptor or ligand; wherein the receptor or ligand forms a dimerization or multimerization structure in a natural signaling pathway to activate or inhibit the signaling pathway, and the second binding domain and the first binding domain target different targets.

2. The fusion protein according to claim 1, wherein the first binding domain targets and binds to an immune checkpoint molecule or a tumor antigen.

3. The fusion protein according to claim 2, wherein the immune checkpoint molecule includes PD-1, PD-L1, CTLA-4, LAG-3, FGL1, TIM-3, Galectin-9, TIGIT, CD155 and CD47; the tumor antigen includes Claudin 18.2, HER-2, Mesothelin, BCMA, SSTR2, GPRC5D, PSMA, FCRH5, CD33, CD123, CD20, A33, CEA, CD28, DLL3, EGFR, VEGFR, VEGFR2, VEGF-A, Nectin-4, FGFR, C-met, RANKL, PDGF, PDGFR, PDGFRα, DLL4, Ang-1 and Ang-2.

4. The fusion protein according to claim 3, wherein the second binding domain targets and binds to human TGF-β, CTLA-4, VEGF, LAGS, CD27, 4-1BB, OX40, CD47, FGL1, TLT-2, CD28, HGF, CSF1, CXCL1, CXCL2, CXCL3, CXCL5, CXCL6, CXCL7, CXCL8, CXCL9, CXCL10, CXCL12, GITR, EGF and ICOSL.

5. The fusion protein according to claim 4, wherein the receptor or ligand is human TGF-β receptor (TGF-βR), CD80, CD86, VEGFR, VEGF-trap, FGL1, CD70, 4-1BBL, OX40L, SIRPα, B7-H3, C-met, CSF1R, CXCR2, CXCR3, CXCR4, GITRL, EGFR and ICOS.

6. The fusion protein according to claim 4, wherein the first binding domain and the second binding domain are selected from the following combinations:
(1) the first binding domain targets and binds to PD-L1, and the second binding domain targets and binds to human TGF-β, VEGF, FGL1, CD47, CD155, HGF, CSF1, CXCL1, CXCL2, CXCL3, CXCL5, CXCL6, CXCL7, CXCL8, CXCL9, CXCL10, CXCL12, EGF or ICOSL; or
(2) the first binding domain targets and binds to PD-1, and the second binding domain targets and binds to human CTLA-4, VEGF, HGF, EGF, CD28, LAG3, CD27, 4-1BB or OX40; or
(3) the first binding domain targets and binds to CTLA-4, and the second binding domain targets and binds to human CD28, VEGF, HGF, EGF, LAG3, CD27, 4-1BB or OX40; or
(4) the first binding domain targets and binds to LAG-3, and the second binding domain targets and binds to human CD28, VEGF, HGF, EGF, CTLA-4, CD27, 4-1BB or OX40; or
(5) the first binding domain targets and binds to FGL1, and the second binding domain targets and binds to human TGF-β, VEGF, CD47, CD155, HGF, CSF1, CXCL1, CXCL2, CXCL3, CXCL5, CXCL6, CXCL7, CXCL8, CXCL9, CXCL10, CXCL12, EGF or ICOSL; or
(6) the first binding domain targets and binds to TIM-3, and the second binding domain targets and binds to human VEGF, HGF, EGF, LAG3, CD27, 4-1BB or OX40; or
(7) the first binding domain targets and binds to Galectin-9, and the second binding domain targets and binds to human TGF-β, VEGF, CD47, CD155, HGF, CSF1, CXCL1, CXCL2, CXCL3, CXCL5, CXCL6, CXCL7, CXCL8, CXCL9, CXCL10, CXCL12, EGF or ICOSL; or
(8) the first binding domain targets and binds to TIGIT, and the second binding domain targets and binds to human TGF-β, HGF, EGF or VEGF; or
(9) the first binding domain targets and binds to CD155, and the second binding domain targets and binds to human TGF-β, VEGF, HGF, EGF, CD47 or CD155; or
(10) the first binding domain targets and binds to Claudin 18.2, HER-2, mesothelin, BCMA, SSTR2, GPRC5D, PSMA, FCRH5, CD33, CD123, CD20, A33, CEA, CD28, DLL3, EGFR, VEGFR, VEGFR2, VEGF-A, Nectin-4, FGFR, C-met, RANKL, PDGF, PDGFR, PDGFRα, DLL-4, Ang-1 or Ang-2, and the second binding domain targets and binds to human CTLA-4, TGF-β, VEGF, FGL1, LAG3, 4-1BB, OX40, CD27, CD28, CD47, CD155, HGF, CSF1, CXCL1, CXCL2, CXCL3, CXCL5, CXCL6, CXCL7, CXCL8, CXCL9, CXCL10, CXCL12, EGF or ICOSL.

7. The fusion protein according to claim 6, wherein the first binding domain and the receptor or ligand are selected from the following combinations:
(1) the first binding domain targets and binds to PD-L1, and the receptor or ligand is selected from TGF-βRII, VEGFR, LAG-3, SIRPα, TIGIT, C-MET, CSF1R, CXCR2, CXCR3, CXCR4, EGFR or ICOS; or
(2) the first binding domain targets and binds to PD-1, and the receptor or ligand is selected from human CD80, CD86, VEGFR, c-MET, EGFR, FGL1, CD70, 4-1BBL or OX40L; or
(3) the first binding domain targets and binds to CTLA-4, and the receptor or ligand is selected from human CD80, CD86, VEGFR, c-MET, EGFR, FGL1, CD70, 4-1BBL or OX40L; or
(4) the first binding domain targets and binds to LAG-3, and the receptor or ligand is selected from human VEGFR, c-MET, EGFR, CD80, CD86, CD70, 4-1BBL or OX40L; or
(5) the first binding domain targets and binds to FGL1, and the receptor or ligand is selected from human TGF-βRII, VEGFR, SIRPα, TIGIT, c-MET, CSF1R, CXCR2, CXCR3, CXCR4, EGFR or ICOS; or
(6) the first binding domain targets and binds to TIM-3, and the receptor or ligand is selected from human VEGFR, c-MET, EGFR, FGL1, CD70, 4-1BBL or OX40L; or
(7) the first binding domain targets and binds to Galectin-9, and the receptor or ligand is selected from human TGF-βRII, VEGFR, SIRPα, TIGIT, c-MET, CSF1R, CXCR2, CXCR3, CXCR4, EGFR or ICOS; or
(8) the first binding domain targets and binds to TIGIT, and the receptor or ligand is selected from human TGF-βRII, c-MET, EGFR or VEGFR; or
(9) the first binding domain targets and binds to CD155, and the receptor or ligand is selected from human TGF-βRII, VEGFR, c-MET, EGFR, SIRPα or TIGIT; or
(10) the first binding domain targets and binds to Claudin 18.2, HER-2, mesothelin, BCMA, SSTR2, GPRC5D, PSMA, FCRH5, CD33, CD123, CD20, A33, CEA, CD28, DLL3, EGFR, VEGFR, VEGFR2, Nectin-4, FGFR, c-MET, RANKL, PDGF, PDGFR, PDGFRa, DLL4, Ang-1 or Ang-2, and the receptor or ligand is selected from human CTLA-4, CD80, CD86, TGF-βRII, VEGFR, FGL1, LAGS, 4-1BB, OX40, CD27, CD28, CD70, c-MET, SIRPα, TIGIT, CSF1R, CXCR2, CXCR3, CXCR4, EGFR or ICOS.

8. The fusion protein according to claim 1, wherein the fragment of the receptor or ligand comprises a fragment of the extracellular region and a fragment of the binding domain of the receptor or ligand.

9. The fusion protein according to claim 7, wherein the first binding domain targets and binds to PD-L1; the second binding domain is a fragment of human TGF-βRII; or the first binding domain targets and binds to PD-1, and the second binding domain is selected from human CD80 ECD, CD80 IgV region, human CD80 IgVIgC, VEGFR1 ECD, VEGFR2 ECD or a combination of the second extracellular region of VEGFR1 and the third extracellular region of VEGFR2; or the first binding domain targets and binds to Claudin 18.2, HER-2 or EGFR, and the second binding domain is selected from human CD80 ECD, CD80 IgV region, human CD80 IgVIgC, VEGFR1 ECD, VEGFR2 ECD or a combination of the second extracellular region of VEGFR1 and the third extracellular region of VEGFR2.

10. The fusion protein according to claim 9, wherein the second binding domain is selected from:
(1) human TGF-βRII comprising the sequence as shown in SEQ ID NO: 31, 131 or 132, or a sequence having an identity of greater than 80%, 85%, 90%, 95%, 96%, 97%, 98%, 99% compared with the sequence as shown in SEQ ID NO: 31, 131 or 132, or an amino acid sequence having 1, 2, 3, 4, 5, 6, 7, 8, 9, or 10 amino acid substitutions or deletions on the sequence as shown in SEQ ID NO: 31, 131 or 132; or
(2) CD80 ECD comprising the sequence as shown in SEQ ID NO: 32 or 133, or a sequence having an identity of greater than 80%, 85%, 90%, 95%, 96%, 97%, 98%, 99% compared with the sequence as shown in SEQ ID NO: 32 or 133, or an amino acid sequence having 1, 2, 3, 4, 5, 6, 7, 8, 9, or 10 amino acid substitutions or deletions on the sequence as shown in SEQ ID NO: 32 or 133; or
(3) a combination of the second extracellular region of VEGFR1 and the third extracellular region of VEGFR2, comprising the sequence as shown in SEQ ID NO: 33, or a sequence having an identity of greater than 80%, 85%, 90%, 95%, 96%, 97%, 98%, 99% compared with the sequence as shown in SEQ ID NO: 33, or an amino acid sequence having 1, 2, 3, 4, 5, 6, 7, 8, 9, or 10 amino acid substitutions or deletions on the sequence as shown in SEQ ID NO: 33.

11. The fusion protein according to claim 9, wherein the first binding domain targets and binds to PD-L1, and the CDRs of the heavy chain variable region and/or the CDRs of the light chain variable region in the Fab of the IgG have the same CDR sequence as the antibody defined by the following sequence, or have 1-2 amino acid substitutions on the CDRs of the antibody defined by the following sequence, wherein the antibody is defined as follows:
(1) the amino acid sequence of the heavy chain variable region is shown in SEQ ID NO: 66; and/or
(2) the amino acid sequence of the light chain variable region is shown in SEQ ID NO: 67.

12. The fusion protein according to claim 11, wherein the CDRs of the heavy chain variable region and/or the CDRs of the light chain variable region in the Fab of the IgG are as follows:
(1) for the heavy chain variable region, the amino acid sequence of CDR1 is selected from SEQ ID NOs: 1-5 and amino acid sequences having 1 or 2 amino acid substitutions on SEQ ID NOs: 1-5; the amino acid sequence of CDR2 is selected from SEQ ID NOs: 6-10 and amino acid sequences having 1 or 2 amino acid substitutions on SEQ ID NOs: 6-10; the amino acid sequence of CDR3 is selected from SEQ ID NOs: 11-15 and amino acid sequences having 1 or 2 amino acid substitutions on SEQ ID NOs: 11-15; and/or
(2) for the light chain variable region, the amino acid sequence of CDR1 is selected from SEQ ID NOs: 16-20 and amino acid sequences having 1 or 2 amino acid substitutions on SEQ ID NOs: 16-20; the amino acid sequence of CDR2 is selected from SEQ ID NOs: 21-25 and amino acid sequences having 1 or 2 amino acid substitutions on SEQ ID NOs: 21-25; the amino acid sequence of CDR3 is selected from SEQ ID NOs: 26-30 and amino acid sequences having 1 or 2 amino acid substitutions on SEQ ID NOs: 26-30.

13. The fusion protein according to claim 12, wherein:
(1) the amino acid sequences of CDRs 1-3 of the heavy chain variable region are SEQ ID NOs: 1, 6, 11, or an amino acid sequence having 1 or 2 amino acid substitutions on SEQ ID NOs: 1, 6, 11; and/or an amino acid sequence of CDRs 1-3 of the light chain variable region are SEQ ID NOs: 16, 21, 26, or an amino acid sequence having 1 or 2 amino acid substitutions on SEQ ID NOs: 16, 21, 26; or
(2) the amino acid sequences of CDRs 1-3 of the heavy chain variable region are SEQ ID NOs: 2, 7, 12, or an amino acid sequence having 1 or 2 amino acid substitutions on SEQ ID NOs: 2, 7, 12; and/or an amino acid sequence of CDRs 1-3 of the light chain variable region are SEQ ID NOs: 17, 22, 27, or an amino acid sequence having 1 or 2 amino acid substitutions on SEQ ID NOs: 17, 22, 27; or
(3) the amino acid sequences of CDRs 1-3 of the heavy chain variable region are SEQ ID NOs: 3, 8, 13, or an amino acid sequence having 1 or 2 amino acid substitutions on SEQ ID NOs: 3, 8, 13; and/or an amino acid sequence of CDRs 1-3 of the light chain variable region are SEQ ID NOs: 18, 23, 28, or an amino acid sequence having 1 or 2 amino acid substitutions on SEQ ID NOs: 18, 23, 28; or
(4) the amino acid sequences of CDRs 1-3 of the heavy chain variable region are SEQ ID NOs: 4, 9, 14, or an amino acid sequence having 1 or 2 amino acid substitutions on SEQ ID NOs: 4, 9, 14; and/or an amino acid sequence of CDRs 1-3 of the light chain variable region are SEQ ID NOs: 19, 24, 29, or an amino acid sequence having 1 or 2 amino acid substitutions on SEQ ID NOs: 19, 24, 29; or
(5) the amino acid sequences of CDRs 1-3 of the heavy chain variable region are SEQ ID NOs: 5, 10, 15, or an amino acid sequence having 1 or 2 amino acid substitutions on SEQ ID NOs: 5, 10, 15; and/or an amino acid sequence of CDRs 1-3 of the light chain variable region are SEQ ID NOs: 20, 25, 30, or an amino acid sequence having 1 or 2 amino acid substitutions on SEQ ID NOs: 20, 25, 30.

14. The fusion protein according to claim 13, wherein the amino acid sequences of CDRs 1-3 of the heavy chain variable region are SEQ ID NOs: 3, 8, 13; and/or an amino acid sequence of CDRs 1-3 of the light chain variable region are SEQ ID NOs: 18, 23, 28.

15. The fusion protein according to claim 14, wherein:
(1) the heavy chain variable region has a sequence as shown in SEQ ID NO: 66, or a sequence that has the same CDRs 1-3 as SEQ ID NO: 66 and has an identity of greater than 80%, 85%, 90%, 95%, 96%, 97%, 98%, 99% compared with SEQ ID NO: 66; and/or
(2) the heavy chain variable region has a sequence as shown in SEQ ID NO: 67, or a sequence that has the same CDRs 1-3 as SEQ ID NO: 67 and has an identity of greater than 80%, 85%, 90%, 95%, 96%, 97%, 98%, 99% compared with SEQ ID NO: 67.

16. The fusion protein according to claim 15, wherein:
(1) the heavy chain of the fusion protein has an amino acid sequence as shown in SEQ ID NO: 72, or a sequence having an identity of greater than 80%, 85%, 90%, 95%, 96%, 97%, 98%, 99% compared with SEQ ID NO: 72;
(2) the light chain of the fusion protein has an amino acid sequence as shown in SEQ ID NO: 73, or a sequence having an identity of greater than 80%, 85%, 90%, 95%, 96%, 97%, 98%, 99% compared with SEQ ID NO: 73.

17. The fusion protein according to claim 16, wherein:
(1) the amino acid sequence of the heavy chain of the fusion protein has 1-15 amino acid site mutations or has an alternative peptide linker compared with SEQ ID NO: 72;
(2) the amino acid sequence of the light chain of the fusion protein has 1-10 amino acid site mutations compared with SEQ ID NO: 73.

18. The fusion protein according to claim 9, wherein the first binding domain targets and binds to PD-1, and the CDRs of the heavy chain variable region and/or the CDRs of the light chain variable region in the Fab of the IgG have the same CDR sequence as the antibody defined by the following sequence, or have 1-2 amino acid substitutions on the CDRs of the antibody defined by the following sequence, wherein the antibody is defined as follows:
(1) the amino acid sequence of the heavy chain variable region is shown in SEQ ID NO: 64; and/or
(2) the amino acid sequence of the light chain variable region is shown in SEQ ID NO: 65.

19. The fusion protein according to claim 18, wherein the CDRs of the heavy chain variable region and/or the CDRs of the light chain variable region in the Fab of the IgG are as follows:
(1) for the heavy chain variable region, the amino acid sequence of CDR1 is selected from SEQ ID NOs: 34-38 and an amino acid sequence having 1 or 2 amino acid substitutions on SEQ ID NOs: 34-38; the amino acid sequence of CDR2 is selected from SEQ ID NOs: 39-43 and an amino acid sequence having 1 or 2 amino acid substitutions on SEQ ID NOs: 39-43; the amino acid sequence of CDR3 is selected from SEQ ID NOs: 44-48 and an amino acid sequence having 1 or 2 amino acid substitutions on SEQ ID NOs: 44-48; and/or
(2) for the light chain variable region, the amino acid sequence of CDR1 is selected from SEQ ID NOs: 49-53 and an amino acid sequence having 1 or 2 amino acid substitutions on SEQ ID NOs: 49-53; the amino acid sequence of CDR2 is selected from SEQ ID NOs: 54-58 and an amino acid sequence having 1 or 2 amino acid substitutions on SEQ ID NOs: 54-58; the amino acid sequence of CDR3 is selected from SEQ ID NOs: 59-63 and an amino acid sequence having 1 or 2 amino acid substitutions on SEQ ID NOs: 59-63.

20. The fusion protein according to claim 19, wherein:
(1) the amino acid sequences of CDRs 1-3 of the heavy chain variable region are SEQ ID NOs: 34, 39, 44, or an amino acid sequence having 1 or 2 amino acid substitutions on SEQ ID NOs: 34, 39, 44; and/or an amino acid sequence of CDRs 1-3 of the light chain variable region are SEQ ID NOs: 49, 54, 59, or an amino acid sequence having 1 or 2 amino acid substitutions on SEQ ID NOs: 49, 54, 59; or
(2) the amino acid sequences of CDRs 1-3 of the heavy chain variable region are SEQ ID NOs: 35, 40, 45, or an amino acid sequence having 1 or 2 amino acid substitutions on SEQ ID NOs: 35, 40, 45; and/or an amino acid sequence of CDRs 1-3 of the light chain variable region are SEQ ID NOs: 50, 55, 60, or an amino acid sequence having 1 or 2 amino acid substitutions on SEQ ID NOs: 50, 55, 60; or
(3) the amino acid sequences of CDRs 1-3 of the heavy chain variable region are SEQ ID NOs: 36, 41, 46, or an amino acid sequence having 1 or 2 amino acid substitutions on SEQ ID NOs: 36, 41, 46; and/or an amino acid sequence of CDRs 1-3 of the light chain variable region are SEQ ID NOs: 51, 56, 61, or an amino acid sequence having 1 or 2 amino acid substitutions on SEQ ID NOs: 51, 56, 61; or
(4) the amino acid sequences of CDRs 1-3 of the heavy chain variable region are SEQ ID NOs: 37, 42, 47, or an amino acid sequence having 1 or 2 amino acid substitutions on SEQ ID NOs: 37, 42, 47; and/or an amino acid sequence of CDRs 1-3 of the light chain variable region are SEQ ID NOs: 52, 57, 62, or an amino acid sequence having 1 or 2 amino acid substitutions on SEQ ID NOs: 52, 57, 62; or
(5) the amino acid sequences of CDRs 1-3 of the heavy chain variable region are SEQ ID NOs: 38, 43, 48, or an amino acid sequence having 1 or 2 amino acid substitutions on SEQ ID NOs: 38, 43, 48; and/or an amino acid sequence of CDRs 1-3 of the light chain variable region are SEQ ID NOs: 53, 58, 63, or an amino acid sequence having 1 or 2 amino acid substitutions on SEQ ID NOs: 53, 58, 63.

21. The fusion protein according to claim 20, wherein the amino acid sequences of CDRs 1-3 of the heavy chain variable region are SEQ ID NOs: 36, 41, 46; and/or an amino acid sequence of CDRs 1-3 of the light chain variable region are SEQ ID NOs: 51, 56, 61.

22. The fusion protein according to claim 21, wherein:
(1) the heavy chain variable region has a sequence as shown in SEQ ID NO: 64, or a sequence that has the same CDRs 1-3 as SEQ ID NO: 64 and has an identity of greater than 80%, 85%, 90%, 95%, 96%, 97%, 98%, 99% compared with SEQ ID NO: 64; and/or
(2) the light chain variable region has a sequence as shown in SEQ ID NO: 65, or a sequence that has the same CDRs 1-3 as SEQ ID NO: 65 and has an identity of greater than 80%, 85%, 90%, 95%, 96%, 97%, 98%, 99% compared with SEQ ID NO: 65.

23. The fusion protein according to claim 22, wherein:
(1) the heavy chain of the fusion protein has an amino acid sequence as shown in SEQ ID NO: 68, or a sequence having an identity of greater than 80%, 85%, 90%, 95%, 96%, 97%, 98%, 99% compared with SEQ ID NO: 68;
(2) the light chain of the fusion protein has an amino acid sequence as shown in SEQ ID NO: 69, or a sequence having an identity of greater than 80%, 85%, 90%, 95%, 96%, 97%, 98%, 99% compared with SEQ ID NO: 69.

24. The fusion protein according to claim 23, wherein:
(1) the amino acid sequence of the heavy chain of the fusion protein has 1-15 amino acid site mutations or has an alternative peptide linker compared with SEQ ID NO: 68;
(2) the amino acid sequence of the light chain of the fusion protein has 1-10 amino acid site mutations compared with SEQ ID NO: 69.

25. The fusion protein according to claim 22, wherein:
(1) the heavy chain of the fusion protein has an amino acid sequence as shown in SEQ ID NO: 70, or a sequence having an identity of greater than 80%, 85%, 90%, 95%, 96%, 97%, 98%, 99% compared with SEQ ID NO: 70;
(2) the light chain of the fusion protein has an amino acid sequence as shown in SEQ ID NO: 71, or a sequence having an identity of greater than 80%, 85%, 90%, 95%, 96%, 97%, 98%, 99% compared with SEQ ID NO: 71.

26. The fusion protein according to claim 25, wherein:
(1) the amino acid sequence of the heavy chain of the fusion protein has 1-15 amino acid site mutations or has an alternative peptide linker compared with SEQ ID NO: 70;
(2) the amino acid sequence of the light chain of the fusion protein has 1-10 amino acid site mutations compared with SEQ ID NO: 71.

27. The fusion protein according to claim 1, wherein the IgG is Atezolizumab, Avelumab, Durvalumab, Nivolumab, Pembrolizumab, Cemiplimab or Ipilimumab.

28. The fusion protein according to claim 1, wherein the C-terminus or/and N-terminus of the second binding domain has a peptide linker, and the peptide linker consists of 2-30 amino acids.

29. The fusion protein of claim 28, wherein the peptide linker is:
(1) (GGGGS)n;
or
(SEQ ID NO: 80)
(2) AKTTPKLEEGEFSEAR;
or
(SEQ ID NO: 81)
(3) AKTTPKLEEGEFSEARV;
or
(SEQ ID NO: 82)
(4) AKTTPKLGG;
or
(SEQ ID NO: 83)
(5) SAKTTPKLGG;
or
(SEQ ID NO: 84)
(6) SAKTTP;
or
(SEQ ID NO: 85)
(7) RADAAP;
or
(SEQ ID NO: 86)
(8) RADAAPTVS;
or
(SEQ ID NO: 87)
(9) RADAAAAGGPGS;
or
(SEQ ID NO: 88)
(10) RADAAAA;
or
(SEQ ID NO: 89)
(11) SAKTTPKLEEGEFSEARV;
or
(SEQ ID NO: 90)
(12) ADAAP;
or
(SEQ ID NO: 91)
(13) DAAPTVSIFPP;
or
(SEQ ID NO: 92)
(14) TVAAP;
or
(SEQ ID NO: 93)
(15) TVAAPSVFIFPP;
or
(SEQ ID NO: 94)
(16) QPKAAP;
or
(SEQ ID NO: 95)
(17) QPKAAPSVTLFPP;
or
(SEQ ID NO: 96)
(18) AKTTPP;
or
(SEQ ID NO: 97)
(19) AKTTPPSVTPLAP;
or
(SEQ ID NO: 98)
(20) AKTTAP;
or
(SEQ ID NO: 99)
(21) AKTTAPSVYPLAP;
or
(SEQ ID NO: 100)
(22) ASTKGP;
or
(SEQ ID NO: 101)
(23) ASTKGPSVFPLAP;
or
(SEQ ID NO: 102)
(24) GENKVEYAPALMALS;
or
(SEQ ID NO: 103)
(25) GPAKELTPLKEAKVS;
or
(SEQ ID NO: 104)
(26) GHEAAAVMQVQYPAS;
or
(SEQ ID NO: 105)
(27) GGGGSGGGGSGGGGSA,
wherein n is equal to 1, 2, 3 or 4.

30. The fusion protein according to claim 1, wherein the size of the inserted second binding domain does not exceed 300 amino acids.

31. The fusion protein according to claim 1, wherein the insertion site of the second binding domain is located in a middle front part of the hinge region, and the insertion site does not affect the formation of disulfide bonds of the immunoglobulin.

32. The fusion protein according to claim 31, wherein the middle front part of the hinge region refers to the part before 231A.

33. The fusion protein according to claim 1, wherein part of the amino acids in the hinge region before and after the insertion site are substituted or deleted.

34. The fusion protein according to claim 33, wherein the hinge region contains D221G and/or C220V mutations.

35. The fusion protein according to claim 1, wherein the IgG is selected from mammalian IgG, humanized IgG, and human IgG, and the mammal includes mouse, rat and rabbit.

36. The fusion protein according to claim 1, wherein the Fc region of the fusion protein is aglycosylated or deglycosylated, or has reduced fucosylation or is afucosylated.

37. An isolated polynucleotide encoding the fusion protein according to claim 1.

38. A nucleic acid construct comprising the polynucleotide according to claim 37.

39. A host cell comprising the polynucleotide according to claim 37.

40. A pharmaceutical composition comprising the fusion protein according to claim 1 and a pharmaceutically acceptable carrier.

41. A method for treating tumor or cancer, comprising the step of administering the fusion protein according to claim 1 to a subject in need of a treatment or relief.

42. A method of treating or preventing a tumor or cancer, comprising administering the fusion protein according to claim 1 to a subject in need thereof.

43. The method according to claim 42, wherein the tumor or cancer includes a solid tumor or a non-solid tumor.

44. A diagnostic kit comprising the fusion protein according to claim 1.

45. A method of manufacturing the fusion protein according to claim 1, comprising culturing a host cell under a condition allowing the expression of a nucleic acid construct comprising an isolated polynucleotide encoding the fusion protein according to claim 1, and recovering the produced fusion protein from the culture, wherein the host cell comprises an isolated polynucleotide encoding the fusion protein according to claim 1.


SEQUENCE LISTING (137 sequences)

SEQ ID NO: 1 - protein, 7 aa, Artificial Sequence
  peptide
GFSLSRY

SEQ ID NO: 2 - protein, 10 aa, Artificial sequence
  Heavy chain variable region CDR1
GFSLSRYSVH

SEQ ID NO: 3 - protein, 5 aa, Artificial sequence
  Heavy chain variable region CDR1
RYSVH

SEQ ID NO: 4 - protein, 6 aa, Artificial sequence
  Heavy chain variable region CDR1
SRYSVH

SEQ ID NO: 5 - protein, 8 aa, Artificial sequence
  Heavy chain variable region CDR1
GFSLSRYS

SEQ ID NO: 6 - protein, 5 aa, Artificial sequence
  Heavy chain variable region CDR2
WGVGT

SEQ ID NO: 7 - protein, 9 aa, Artificial sequence
  Heavy chain variable region CDR2
MIWGVGTTD

SEQ ID NO: 8 - protein, 16 aa, Artificial sequence
  Heavy chain variable region CDR2
MIWGVGTTDYNSALKS

SEQ ID NO: 9 - protein, 12 aa, Artificial sequence
  Heavy chain variable region CDR2
WLGMIWGVGTTD

SEQ ID NO: 10 - protein, 7 aa, Artificial sequence
  Heavy chain variable region CDR2
IWGVGTT

SEQ ID NO: 11 - protein, 10 aa, Artificial sequence
  Heavy chain variable region CDR3
NWGTADYFDY

SEQ ID NO: 12 - protein, 10 aa, Artificial sequence
  Heavy chain variable region CDR3
NWGTADYFDY

SEQ ID NO: 13 - protein, 10 aa, Artificial sequence
  Heavy chain variable region CDR3
NWGTADYFDY

SEQ ID NO: 14 - protein, 11 aa, Artificial sequence
  Heavy chain variable region CDR3
ARNWGTADYFD

SEQ ID NO: 15 - protein, 12 aa, Artificial sequence
  Heavy chain variable region CDR3
ARNWGTADYFDY

SEQ ID NO: 16 - protein, 15 aa, Artificial sequence
  Light chain variable region CDR1
RASKSVHTSGYSYMH

SEQ ID NO: 17 - protein, 15 aa, Artificial sequence
  Light chain variable region CDR1
RASKSVHTSGYSYMH

SEQ ID NO: 18 - protein, 15 aa, Artificial sequence
  Light chain variable region CDR1
RASKSVHTSGYSYMH

SEQ ID NO: 19 - protein, 11 aa, Artificial sequence
  Light chain variable region CDR1
HTSGYSYMHWY

SEQ ID NO: 20 - protein, 10 aa, Artificial sequence
  Light chain variable region CDR1
KSVHTSGYSY

SEQ ID NO: 21 - protein, 7 aa, Artificial sequence
  Light chain variable region CDR2
LASNLES

SEQ ID NO: 22 - protein, 7 aa, Artificial sequence
  Light chain variable region CDR2
LASNLES

SEQ ID NO: 23 - protein, 7 aa, Artificial sequence
  Light chain variable region CDR2
LASNLES

SEQ ID NO: 24 - protein, 10 aa, Artificial sequence
  Light chain variable region CDR2
LLIYLASNLE

SEQ ID NO: 25 - protein, 3 aa, Artificial sequence
  Light chain variable region CDR2
LAS

SEQ ID NO: 26 - protein, 9 aa, Artificial sequence
  Light chain variable region CDR3
QHSGELPYT

SEQ ID NO: 27 - protein, 9 aa, Artificial sequence
  Light chain variable region CDR3
QHSGELPYT

SEQ ID NO: 28 - protein, 9 aa, Artificial sequence
  Light chain variable region CDR3
QHSGELPYT

SEQ ID NO: 29 - protein, 8 aa, Artificial sequence
  Light chain variable region CDR3
QHSGELPY

SEQ ID NO: 30 - protein, 9 aa, Artificial sequence
  Light chain variable region CDR3
QHSGELPYT

SEQ ID NO: 31 - protein, 137 aa, Artificial sequence
  peptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 32 - protein, 198 aa, Artificial sequence
  CD80?ECD
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWN

SEQ ID NO: 33 - protein, 202 aa, Artificial sequence
  VEGFR
GRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEP

SEQ ID NO: 34 - protein, 7 aa, Artificial sequence
  Heavy chain variable region CDR1
GITFSNS

SEQ ID NO: 35 - protein, 10 aa, Artificial sequence
  Heavy chain variable region CDR1
GITFSNSGMH

SEQ ID NO: 36 - protein, 5 aa, Artificial sequence
  Heavy chain variable region CDR1
NSGMH

SEQ ID NO: 37 - protein, 6 aa, Artificial sequence
  Heavy chain variable region CDR1
SNSGMH

SEQ ID NO: 38 - protein, 8 aa, Artificial sequence
  Heavy chain variable region CDR1
GITFSNSG

SEQ ID NO: 39 - protein, 6 aa, Artificial sequence
  Heavy chain variable region CDR2
WYDGSK

SEQ ID NO: 40 - protein, 10 aa, Artificial sequence
  Heavy chain variable region CDR2
VIWYDGSKRY

SEQ ID NO: 41 - protein, 17 aa, Artificial sequence
  Heavy chain variable region CDR2
VIWYDGSKRYYADSVKG

SEQ ID NO: 42 - protein, 13 aa, Artificial sequence
  Heavy chain variable region CDR2
WVAVIWYDGSKRY

SEQ ID NO: 43 - protein, 8 aa, Artificial sequence
  Heavy chain variable region CDR2
IWYDGSKR

SEQ ID NO: 44 - protein, 4 aa, Artificial sequence
  Heavy chain variable region CDR3
NDDY

SEQ ID NO: 45 - protein, 4 aa, Artificial sequence
  Heavy chain variable region CDR3
NDDY

SEQ ID NO: 46 - protein, 4 aa, Artificial sequence
  Heavy chain variable region CDR3
NDDY

SEQ ID NO: 47 - protein, 5 aa, Artificial sequence
  Heavy chain variable region CDR3
ATNDD

SEQ ID NO: 48 - protein, 6 aa, Artificial sequence
  Heavy chain variable region CDR3
ATNDDY

SEQ ID NO: 49 - protein, 11 aa, Artificial sequence
  Light chain variable region CDR1
RASQSVSSYLA

SEQ ID NO: 50 - protein, 11 aa, Artificial sequence
  Light chain variable region CDR1
RASQSVSSYLA

SEQ ID NO: 51 - protein, 11 aa, Artificial sequence
  Light chain variable region CDR1
RASQSVSSYLA

SEQ ID NO: 52 - protein, 7 aa, Artificial sequence
  Light chain variable region CDR1
SSYLAWY

SEQ ID NO: 53 - protein, 6 aa, Artificial sequence
  Light chain variable region CDR1
QSVSSY

SEQ ID NO: 54 - protein, 7 aa, Artificial sequence
  Light chain variable region CDR2
DASNRAT

SEQ ID NO: 55 - protein, 7 aa, Artificial sequence
  Light chain variable region CDR2
DASNRAT

SEQ ID NO: 56 - protein, 7 aa, Artificial sequence
  Light chain variable region CDR2
DASNRAT

SEQ ID NO: 57 - protein, 10 aa, Artificial sequence
  Light chain variable region CDR2
LLIYDASNRA

SEQ ID NO: 58 - protein, 3 aa, Artificial sequence
  Light chain variable region CDR2
DAS

SEQ ID NO: 59 - protein, 9 aa, Artificial sequence
  Light chain variable region CDR3
QQSSNWPRT

SEQ ID NO: 60 - protein, 9 aa, Artificial sequence
  Light chain variable region CDR3
QQSSNWPRT

SEQ ID NO: 61 - protein, 9 aa, Artificial sequence
  Light chain variable region CDR3
QQSSNWPRT

SEQ ID NO: 62 - protein, 8 aa, Artificial sequence
  Light chain variable region CDR3
QQSSNWPR

SEQ ID NO: 63 - protein, 9 aa, Artificial sequence
  Light chain variable region CDR3
QQSSNWPRT

SEQ ID NO: 64 - protein, 113 aa, Artificial sequence [sequence omitted]

SEQ ID NO: 65 - protein, 107 aa, Artificial sequence [sequence omitted]

SEQ ID NO: 66 - protein, 120 aa, Artificial sequence
  Heavy chain variable region amino acid sequence
QVQLQESGPGLVKPSETLSLTCTVSGFSLSRYSVHWIRQPPGKGLEWLGMIWGVGTTDYNSALKSRLTISKDTSKNQFSLKLSSVTAADTAVYYCARNWGTADYFDYWGQGTTVTVSSAS

SEQ ID NO: 67 - protein, 113 aa, Artificial sequence
  Light chain variable region amino acid sequence
DIVLTQSPASLAVSPGQRATITCRASKSVHTSGYSYMHWYQQKPGQPPKLLIYLASNLESGVPARFSGSGSGTDFTLTINPVEANDTANYYCQHSGELPYTFGGGTKVEIKRT

SEQ ID NO: 68 - protein, 668 aa, Artificial sequence
  Hib-PDC heavy chain amino acid sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNGGGGSGGGGSGGGGSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 214 aa, Artificial sequence
  Hib-PDC light chain amino acid sequence
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 672 aa, Artificial sequence
  Hib-PDV heavy chain amino acid sequence
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESGGGGSGGGGSGGGGSGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEPGGGGSGGGGSGGGGSKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 214 aa, Artificial sequence; identical to SEQ ID NO: 69
  Hib-PDV light chain amino acid sequence

SEQ ID NO: 72 - protein, 615 aa, Artificial sequence
  Hib-PLT heavy chain amino acid sequence
QVQLQESGPGLVKPSETLSLTCTVSGFSLSRYSVHWIRQPPGKGLEWLGMIWGVGTTDYNSALKSRLTISKDTSKNQFSLKLSSVTAADTAVYYCARNWGTADYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 218 aa, Artificial sequence
  Hib-PLT light chain amino acid sequence
DIVLTQSPASLAVSPGQRATITCRASKSVHTSGYSYMHWYQQKPGQPPKLLIYLASNLESGVPARFSGSGSGTDFTLTINPVEANDTANYYCQHSGELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 2007 bp, Artificial sequence
  Hib-PDC heavy chain nucleic acid sequence
caggtgcagctcgtggagagcgggggaggcgtcgtccagcctggccgtagcctgcggctggactgtaaggctagcggaatcacgttcagcaacagcggaatgcactgggtcagacaggctcctggcaaaggcctcgaatgggtcgccgtgatctggtacgacgggagcaagagatactacgcagattcagtgaagggccgtttcacaatcagccgtgacaactcgaagaacacactgttcctgcagatgaacagcctgagggcagaagacacagcagtctactactgcgctacaaatgacgactactggggccagggaacactggtcaccgtgagctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccggtggcggcggaagcggcggcggaggcagcggcggaggcggcagcgtgatccatgtcacgaaggaggtcaaagaagtggccaccctcagctgcggtcacaacgtcagcgtggaagagttggcccagaccagaatctactggcagaaggagaagaagatggtcttgaccatgatgagcggcgacatgaacatctggccagagtacaagaatagaactatcttcgacatcaccaataacctgagcatcgtgatcctcgctttgcgtccgagcgacgaaggcacctacgagtgcgtagtgctaaagtacgagaaagacgccttcaagcgggagcacctggcagaagtaaccctgagcgtgaaggcagacttcccaacgcctagcatctccgacttcgagatccccacaagcaacatccggcggatcatctgtagcacctccgggggtttccccgagcctcacctgtcctggctcgagaacggcgaggagctgaacgccatcaacaccaccgtgagccaggaccccgagacagaactgtacgccgtgagctccaagctcgacttcaacatgaccacaaatcacagcttcatgtgcctcatcaagtacggacacctgcgggtgaatcagacgttcaactggaacggcggggggggtagcggcggcgggggctcaggtggggggggctcaaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatag

SEQ ID NO: 75 - DNA, 645 bp, Artificial sequence
  Hib-PDC light chain nucleic acid sequence
gagatcgtgctgacacagagcccagccactctgtcactgtccccaggagaaagggctactctgtcttgccgggcaagccagtctgtctccagctacctggcctggtatcagcagaagcccggacaggctcctagactgctgatctacgacgcaagtaacagagccaccggcatccccgcacgcttcagtggctcaggctccggaacagactttactctgaccatctctagtctggagcctgaagatttcgccgtgtactattgtcagcagagctctaattggcctagaaccttcggccagggcaccaaagtcgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 76 - DNA, 2019 bp, Artificial sequence
  Hib-PDV heavy chain nucleic acid sequence
caggtgcagctcgtggagagcgggggaggcgtcgtccagcctggccgtagcctgcggctggactgtaaggctagcggaatcacgttcagcaacagcggaatgcactgggtcagacaggctcctggcaaaggcctcgaatgggtcgccgtgatctggtacgacgggagcaagagatactacgcagattcagtgaagggccgtttcacaatcagccgtgacaactcgaagaacacactgttcctgcagatgaacagcctgagggcagaagacacagcagtctactactgcgctacaaatgacgactactggggccagggaacactggtcaccgtgagctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccggtggcggcggaagcggcggcggaggcagcggcggaggcggcagcggtagaccattcgtagagatgtacagcgaaatccccgaaatcatccacatgactgaaggaagggagctcgtcatcccctgccgggttacgtcacctaacatcactgttactctgaagaagttcccactcgacactttgatccctgatggaaaacgcatcatctgggacagcagaaagggcttcatcatctcaaatgcaacgtacaaagaaatcggactcctgacctgtgaagcaacagtcaatggacatttgtataagacaaactatctcacacatcgacagaccaatacaatcatcgatgtggttctgagcccgtctcatggaatcgaactatctgttggagaaaagctcgtcctgaattgtacagcaagaactgaactgaatgtgggaatcgacttcaactgggaatacccttcttcgaagcatcagcataagaaactcgtaaaccgagacctaaagacccagtctgggagcgagatgaagaaattcttgagcaccctgactatcgatggtgtaacccggagcgaccagggattgtacacctgtgcagcatccagcgggctgatgaccaagaagaacagcacattcgtcagggtccatgaacccggcggggggggtagcggcggcgggggctcaggtggggggggctcaaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatag

SEQ ID NO: 77 - DNA, 645 bp, Artificial sequence; identical to SEQ ID NO: 75
  Hib-PDV light chain nucleic acid sequence

SEQ ID NO: 78 - DNA, 1848 bp, Artificial sequence
  Hib-PLT heavy chain nucleic acid sequence
caggtgcagctgcaggagtccggaccaggactggtgaagccatccgagaccctgagcctgacctgtacagtgtccggcttcagcctgtctaggtacagcgtgcactggatcagacagccacctggcaagggactggagtggctgggcatgatctggggcgtgggcaccacagactacaactctgctctgaagtccagactgaccatcagcaaggatacatctaagaatcagttcagcctgaagctgtccagcgtgaccgccgctgacacagccgtgtactattgcgctcgcaactggggcaccgccgactacttcgactattggggccagggcaccacagtgacagtgtcttccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaggcgggggaggcagcggcggcggaggaagcgggggcggaggtagcaccatccctccacacgtgcagaagagcgtgaacaatgacatgatcgtcaccgacaacaacggcgccgtcaagttcccccagctgtgcaaattctgcgacgtgaggttctccacgtgcgacaaccagaagagctgtatgagcaactgcagcatcacatccatctgcgaaaaaccccaggaagtgtgcgtcgccgtctggcggaagaacgacgagaacatcacactggagaccgtgtgccacgaccccaaactgccctaccacgacttcatcctggaggacgccgccagcccaaagtgcatcatgaaagagaagaagaagccgggcgagactttcttcatgtgctcctgcagctccgacgagtgcaacgataatatcatcttcagcgaagaatacaacacatctaacccagacggagggggcggatccgggggcggcggaagcggcggggggggcagcactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 79 - DNA, 657 bp, Artificial sequence
  Hib-PLT light chain nucleic acid sequence
gatatcgtgctgacccagtctccagcttccctggccgtgtccccaggacagagggccaccatcacatgtcgggcttccaagagcgtgcacacaagcggctactcttatatgcattggtaccagcagaagcccggccagccccctaagctgctgatctatctggcttccaacctggagagcggagtgccagctaggttctctggctccggcagcggcaccgactttaccctgacaatcaatcctgtggaggccaacgatacagctaattactattgccagcactccggagagctgccatacaccttcggcggaggcacaaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 80 - protein, 16 aa, Artificial sequence
  Peptide linker
AKTTPKLEEGEFSEAR

SEQ ID NO: 81 - protein, 17 aa, Artificial sequence
  Peptide linker
AKTTPKLEEGEFSEARV

SEQ ID NO: 82 - protein, 9 aa, Artificial sequence
  Peptide linker
AKTTPKLGG

SEQ ID NO: 83 - protein, 10 aa, Artificial sequence
  Peptide linker
SAKTTPKLGG

SEQ ID NO: 84 - protein, 6 aa, Artificial sequence
  Peptide linker
SAKTTP

SEQ ID NO: 85 - protein, 6 aa, Artificial sequence
  Peptide linker
RADAAP

SEQ ID NO: 86 - protein, 9 aa, Artificial sequence
  Peptide linker
RADAAPTVS

SEQ ID NO: 87 - protein, 12 aa, Artificial sequence
  Peptide linker
RADAAAAGGPGS

SEQ ID NO: 88 - protein, 7 aa, Artificial sequence
  Peptide linker
RADAAAA

SEQ ID NO: 89 - protein, 18 aa, Artificial sequence
  Peptide linker
SAKTTPKLEEGEFSEARV

SEQ ID NO: 90 - protein, 5 aa, Artificial sequence
  Peptide linker
ADAAP

SEQ ID NO: 91 - protein, 11 aa, Artificial sequence
  Peptide linker
DAAPTVSIFPP

SEQ ID NO: 92 - protein, 5 aa, Artificial sequence
  Peptide linker
TVAAP

SEQ ID NO: 93 - protein, 12 aa, Artificial sequence
  Peptide linker
TVAAPSVFIFPP

SEQ ID NO: 94 - protein, 6 aa, Artificial sequence
  Peptide linker
QPKAAP

SEQ ID NO: 95 - protein, 13 aa, Artificial sequence
  Peptide linker
QPKAAPSVTLFPP

SEQ ID NO: 96 - protein, 6 aa, Artificial sequence
  Peptide linker
AKTTPP

SEQ ID NO: 97 - protein, 13 aa, Artificial sequence
  Peptide linker
AKTTPPSVTPLAP

SEQ ID NO: 98 - protein, 6 aa, Artificial sequence
  Peptide linker
AKTTAP

SEQ ID NO: 99 - protein, 13 aa, Artificial sequence
  Peptide linker
AKTTAPSVYPLAP

SEQ ID NO: 100 - protein, 6 aa, Artificial sequence
  Peptide linker
ASTKGP

SEQ ID NO: 101 - protein, 13 aa, Artificial sequence
  Peptide linker
ASTKGPSVFPLAP

SEQ ID NO: 102 - protein, 15 aa, Artificial sequence
  Peptide linker
GENKVEYAPALMALS

SEQ ID NO: 103 - protein, 15 aa, Artificial sequence
  Peptide linker
GPAKELTPLKEAKVS

SEQ ID NO: 104 - protein, 15 aa, Artificial sequence
  Peptide linker
GHEAAAVMQVQYPAS

SEQ ID NO: 105 - protein, 16 aa, Artificial sequence
  Peptide linker
GGGGSGGGGSGGGGSA

SEQ ID NO: 106 - protein, 448 aa, Artificial sequence
  Heavy chain amino acid sequence of Atezolizumab
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 214 aa, Artificial sequence
  Light chain amino acid sequence of Atezolizumab
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 450 aa, Artificial sequence
  Heavy chain amino acid sequence of Avelumab
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 216 aa, Artificial sequence
  Light chain amino acid sequence of Avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 110 - protein, 451 aa, Artificial sequence
  Heavy chain amino acid sequence of Durvalumab
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 215 aa, Artificial sequence
  Light chain amino acid sequence of Durvalumab
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 440 aa, Artificial sequence
  Heavy chain amino acid sequence of Nivolumab
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 214 aa, Artificial sequence; identical to SEQ ID NO: 69
  Light chain amino acid sequence of Nivolumab

SEQ ID NO: 114 - protein, 447 aa, Artificial sequence
  Pembrolizumab heavy chain amino acid sequence
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 218 aa, Artificial sequence
  Pembrolizumab light chain amino acid sequence
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 448 aa, Artificial sequence
  Ipilimumab heavy chain amino acid sequence
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 215 aa, Artificial sequence
  Ipilimumab light chain amino acid sequence
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 444 aa, Artificial sequence
  Cemiplimab heavy chain amino acid sequence
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - protein, 214 aa, Artificial sequence
  Cemiplimab light chain amino acid sequence
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 5 aa, Artificial sequence
  Peptide linker
GGGGS

SEQ ID NO: 121 - protein, 10 aa, Artificial sequence
  Peptide linker
GGGGSGGGGS

SEQ ID NO: 122 - protein, 15 aa, Artificial sequence
  Peptide linker
GGGGSGGGGSGGGGS

SEQ ID NO: 123 - protein, 20 aa, Artificial sequence
  Peptide linker
GGGGSGGGGSGGGGSGGGGS

SEQ ID NO: 124 - protein, 600 aa, Artificial sequence
  Heavy chain amino acid sequence of Control protein 1
QVQLQESGPGLVKPSETLSLTCTVSGFSLSRYSVHWIRQPPGKGLEWLGMIWGVGTTDYNSALKSRLTISKDTSKNQFSLKLSSVTAADTAVYYCARNWGTADYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 125 - protein, 218 aa, Artificial sequence; identical to SEQ ID NO: 73
  Light chain amino acid sequence of Control protein 1

SEQ ID NO: 126 - protein, 653 aa, Artificial sequence
  Heavy chain amino acid sequence of Control protein 2
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWN

SEQ ID NO: 127 - protein, 214 aa, Artificial sequence; identical to SEQ ID NO: 69
  Light chain amino acid sequence of Control protein 2

SEQ ID NO: 128 - protein, 657 aa, Artificial sequence
  Heavy chain amino acid sequence of Control protein 3
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEP

SEQ ID NO: 129 - protein, 214 aa, Artificial sequence; identical to SEQ ID NO: 69
  Light chain amino acid sequence of Control protein 3

SEQ ID NO: 130 - protein, 202 aa, Artificial sequence; identical to SEQ ID NO: 33
  VEGF-R fragment

SEQ ID NO: 131 - protein, 117 aa, Artificial sequence
  peptide
GAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 132 - protein, 115 aa, Artificial sequence
  peptide
VKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 133 - protein, 112 aa, Artificial sequence
  CD80?ECD
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPS

SEQ ID NO: 134 - protein, 288 aa, Artificial sequence
  CD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 135 - protein, 256 aa, Artificial sequence
  CD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTSFAPRCRERRRNERLRRESVRPV

SEQ ID NO: 136 - protein, 162 aa, Artificial sequence
  CD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKGFAPRCRERRRNERLRRESVRPV

SEQ ID NO: 137 - protein, 137 aa, Artificial sequence; identical to SEQ ID NO: 31
  peptide